(19) United States
(12) Patent Application Publication
(10) Pub. No.: US 2026/0000425 A1
(43) Pub. Date: Jan. 1, 2026

(54) CLOT EXTRACTION DEVICE CONTROLLER AND RELATED METHODS

(71) Applicant: Asahi Intecc Co., Ltd., Seto-shi (JP)

(72) Inventors: Christian TOSTADO, Temecula, CA (US); Jeff BURKE, Fallbrook, CA (US); Hsing-ching Crystal HSU, Santa Ana, CA (US); Elijah De GUZMAN, Irvine, CA (US); Michael LUK, Orange, CA (US)

(73) Assignee: Asahi Intecc Co., Ltd., Seto-shi (JP)

(21) Appl. No.: 19/247,422

(22) Filed: Jun. 24, 2025

Related U.S. Application Data
(60) Provisional application No. 63/665,422, filed on Jun. 28, 2024.

(51) Int. Cl. A61B 17/3207; A61B 17/00; A61B 17/22; A61B 17/221

(52) U.S. Cl. CPC A61B 17/320725; A61B 17/221; A61B 2017/00477; A61B 2017/22038; A61B 2017/22079; A61B 2017/2215; A61B 2217/007

(57) ABSTRACT

Systems and apparatuses are provided for clot extraction. In a first aspect, a system includes a first shaft, a second shaft, a third shaft, a coring element including a blade, and a mesh. The coring element has a first end and a second end. The first end of the coring element is coupled to the first shaft and the second end of the coring element is coupled to the second shaft. The mesh includes an open end and a second end. The open end of the mesh is coupled to a proximal portion of the coring element and the second end of the mesh is coupled to the third shaft through a tip structure. Other aspects and features are also claimed and described.

### CROSS-REFERENCE TO RELATED APPLICATION

The present application claims priority to U.S. Provisional Ser. No. 63/665,422 filed on Jun. 28, 2024, the entire contents of each of which are incorporated herein by reference.

### FIELD OF INVENTION

The present application relates generally to the medical field. More specifically, the present application relates to thrombectomy devices and procedures.

### BACKGROUND

Thrombectomy is a medical procedure for removing blood clots from blood vessels, particularly within the brain or other vital organs, and is commonly employed to treat conditions such as ischemic stroke, deep vein thrombosis (DVT), and pulmonary embolism. The procedure involves the use of specialized devices, often guided by imaging techniques such as fluoroscopy or angiography, to physically extract or dissolve the blood clots. For instance, a delivery catheter may be navigated through the vasculature to reach the site of the blood clot. Specialized devices, such as a stent retriever housed within the delivery catheter and/or an aspiration catheter, are then employed to physically remove or break down the blood clot. Stent retrievers, for example, are designed to trap and remove the clot when deployed within the vessel.

### SUMMARY

The following summarizes some embodiments of the present disclosure to provide a basic understanding of the discussed technology. This summary is not an extensive overview of all contemplated features of the disclosure and is intended neither to identify key or critical elements of all embodiments of the disclosure nor to delineate the scope of any or all embodiments of the disclosure. Its sole purpose is to present some concepts of one or more embodiments of the disclosure in summary form as a prelude to the more detailed description that is presented later.

A new and innovative vascular introducer set is provided that includes a dilator having a rotation mechanism for deploying a funnel of an introducer sheath. Deploying the funnel via a rotation mechanism allows for deploying the funnel gradually and safely. The funnel may have a shorter length than conventional funnels, which increases treatment length. When the vascular introducer set is first inserted into a vascular system of a patient, the funnel is in a constrained state at least partially within a cap of the dilator. Once the introducer sheath is seated at a desired location, the funnel can be released by rotating a first component of the rotation mechanism to cause the cap to advance away from the introducer sheath. When the cap no longer constrains the funnel, the funnel is released into an expanded state.

The dilator can then be removed from the vascular system. The first component may be rotated in the opposite direction to retract the cap toward the introducer sheath prior to withdrawing the dilator out of, or from, the introducer sheath. At this point, the introducer sheath is set, or positioned, in place to aid in the introduction of a device into the vascular system of the patient. For example, a new and innovative extractor device provided herein can be introduced into the vascular system of the patient through the introducer sheath, though other devices may be introduced.

The present disclosure includes embodiments of an introducer set, which includes a tube (e.g., an introducer sheath) and a distally-facing receiver (e.g., a braided tubular structure, such as a braided funnel) as well as an atraumatic distal element (e.g., a dilator), and embodiments of an extractor device, which includes a control handle and a clot extraction device (e.g., a device that includes a coring element (which may comprise a multi-arm coring element, such as a coring element that is resilient or self-expanding and/or comprises a central region/portion/segment connected (e.g., integrally) to multiple arms, each of which may extend from the central segment) and a mesh element that is proximally-facing and used to capture clot material (such as that dislodged from a vessel wall by the coring element) as it is withdrawn proximally, the coring and mesh elements together being characterizable as an extractor catheter), each of which separately, and all of which together, are useful for clot removal. Some embodiments of the present systems include both an introducer set (including any of those described and/or depicted in this disclosure) and an extractor device (including any of those described and/or depicted in this disclosure).

In an example, a system, which may be characterized as an introducer or an introducer set, includes an introducer sheath and a dilator. The introducer sheath includes a tube defining a lumen, and a self-expanding funnel coupled to the tube. The dilator is disposed through the lumen and includes a cap and an adjustment system. At least a portion of the self-expanding funnel is disposed within the cap such that the self-expanding funnel is maintained in a constrained state by the cap. The adjustment system is structured such that rotating a first component of the adjustment system about an axis that extends through the cap advances the cap away from the introducer sheath such that the self-expanding funnel is unconstrained and able to transition to an expanded state.

In another example, a method includes inserting an introducer set in a pre-deployment state into a vascular system of a patient. The introducer set includes an introducer sheath and a dilator. The introducer sheath includes a tube defining a lumen, and a self-expanding funnel coupled to the tube. The dilator is disposed through the lumen and includes a cap and an adjustment system. At least a portion of the self-expanding funnel is disposed within the cap in the pre-deployment state such that the self-expanding funnel is maintained in a constrained state by the cap. In some embodiments, the portion of the funnel that extends away from the tube is not covered and/or does not include a coating and/or a membrane. In some embodiments, the portion of the funnel that extends away from the tube is not covered and/or does not include a coating and/or a membrane. The adjustment system is structured to advance the cap away from the tube. The method further includes rotating a first component of the adjustment system about an axis that extends through the cap such that the cap advances away from the introducer sheath, thereby transitioning the introducer set to a deployed state in which the self-expanding funnel is unconstrained to allow the self-expanding funnel to transition to an expanded state. In some embodiments, the funnel is sized relative to the vessel in which it will be positioned such that, after transitioning to the deployed state, a distal portion of the funnel contacts the vessel. In some further embodiments, the distal portion of the funnel seals against the vessel when or as a vacuum is applied through the lumen. The method further includes withdrawing the dilator through the lumen and out of the vascular system of the patient.

In another example, a dilator includes an outer shaft including at least one exterior thread along a proximal end of the outer shaft; an inner shaft disposed through the outer shaft; a cap coupled to a distal end of the inner shaft; and an adjusting member coupled to a proximal end of the inner shaft. The adjusting member includes at least one interior thread engaged with the at least one exterior thread of the outer shaft. Rotating the adjusting member relative to the outer shaft translates the cap relative to the outer shaft.

The new and innovative extractor device includes a controller that includes a control handle (sometimes referred to herein as a handle) having a 3-shaft assembly that includes an inner shaft, a middle shaft, and an outer shaft. These shafts are hollow and may be referred to as tubes. While the outer shaft is fixed translationally to a housing of the control handle, the inner shaft and the middle shaft may translate relative to the housing based on an adjustment system of the control handle. For example, the adjustment system may include a depressible trigger that restricts or allows movement of the inner shaft. In another example, the adjustment system may include a depressible and rotatable dial that restricts, allows, or controls movement of the middle shaft. In at least some embodiments, the adjustment system may include a knob that may be rotated to rotate each of the inner shaft, the middle shaft, and the outer shaft about a central axis of the 3-shaft assembly.

An example of a method of controlling a shaft assembly for a medical procedure comprises restricting movement of an inner shaft of the shaft assembly by, in part, biasing a trigger to interfere with movement of a sliding member coupled to the inner shaft; depressing the trigger and manually translating the sliding member from a location proximal of a housing into which the sliding member distally extends and through which the inner shaft is positioned; restricting movement of a middle shaft of the shaft assembly by, in part, biasing an operator-controlled engagement portion (e.g., of, or coupled to, a pivoting member, such as a dial) to be disengaged with an engagement portion (e.g., of, or coupled to, a hub) positioned in the housing and coupled to the middle shaft; and engaging a portion of the operator-controlled engagement with the engagement portion positioned in the housing and causing (e.g., by rotating or pivoting the pivoting member) the middle shaft to translate. The method may also include rotating the inner shaft, the middle shaft, and an outer shaft of the shaft assembly together, including after disengaging a knob from the housing and by thereafter rotating the knob, which causes (including indirectly) rotation of the outer shaft, which causes (including indirectly) rotation of the middle shaft, which causes (including indirectly) rotation of the inner shaft.

An example flushing method includes collapsing an extractor catheter; introducing flushing solution (e.g., saline solution) into a first flush port of a control handle coupled to the extractor catheter to flush space between an inner shaft and a middle shaft; introducing flushing solution into a second flush port to flush space between the middle shaft and an outer shaft; introducing flushing solution into a third flush port to flush space between the outer shaft and a cover sleeve; advancing the cover sleeve to sheath the extractor catheter; and introducing flushing solution into a fourth flush port to flush space between a guidewire and the inner shaft.

The extractor device further includes an extractor catheter coupled to the control handle. More specifically, the extractor catheter is coupled to the 3-shaft assembly. The extractor catheter includes a coring element and a mesh, or mesh element, coupled to the coring element at a proximal, open end of the mesh element. The mesh may be more broadly referred to as a clot-capturing element. A distal end of the mesh element is coupled to the inner shaft. A proximal portion of the coring element, which is distal to the proximal end of the coring element, is coupled to the translationally fixed outer shaft, which serves as an anchor for the coring element. A distal end of the coring element is coupled to the middle shaft. In this way, a physician may operate the adjustment mechanism of the control handle to manipulate a shape of the extractor catheter or to allow the extractor catheter to adjust freely. The coring element may comprise a multi-arm coring element, such as a coring element that is resilient or self-expanding and/or comprises a central region/portion/segment connected (e.g., integrally) to multiple arms, each of which may extend from the central segment.

The coring element includes a blade or cutting edge configured to physically separate a blood clot from a blood vessel wall or break down a blood clot within a blood vessel. The separated or broken down clot can be captured within the mesh element. The proximal end of the coring element, and thereby the open end of the mesh element, is disposed at an obtuse angle relative to an axis (e.g., a central axis) of the telescoping shaft assembly, which allows for a larger opening to collect clot material than if the open end were disposed perpendicular to the axis. The obtuse angle may change as a shape of the coring element is adjusted. In at least some embodiments, the opening remains approximately the same size no matter the obtuse angle.

In an example, a clot extraction device controller includes a first (e.g., inner) shaft, a second (e.g., middle or outer) shaft disposed (e.g., concentrically) around a portion of the first shaft, and a control handle. The control handle includes a housing, a sliding member disposed through the housing and coupled to the first shaft, a first hub disposed within the housing and coupled to the second shaft, and a first control member (e.g., a trigger) coupled to the housing. The sliding member is disposed through a portion of the first hub. The first control member is configured and coupled to the housing such that the first control member is movable between a first position in which the first control member restricts movement of the sliding member relative to the housing and a second position in which the first control member allows movement of the sliding member relative to the housing.

In another example, a clot extraction device controller includes a first (e.g., inner) shaft, a second (e.g., middle) shaft disposed (e.g., concentrically) around a portion of the first shaft, and a control handle. The control handle includes a housing, a sliding member disposed through the housing and coupled to the first shaft, a first hub disposed within the housing and coupled to the second shaft, and a first control member (e.g., a dial) coupled to the housing. The sliding member is disposed through a portion of the first hub. The first control member is configured and coupled to the housing such that the first control member is movable between a first position in which the first control member allows movement of the first hub relative to the housing and a second position in which the first control member restricts movement of the first hub relative to the housing.

In another example, a clot extraction device control handle includes a control member (e.g., a knob), a sliding member configured to be coupled to an inner shaft, a first hub configured to be coupled to a middle shaft disposed (e.g., concentrically) around a portion of the inner shaft, and a second hub configured to be coupled to an outer shaft disposed (e.g., concentrically) around a portion of middle shaft. Each of the control member, the first hub, and the second hub are rotationally coupled to the sliding member such that rotating the control member rotates each of the sliding member, the first hub, and the second hub.

In another example, a system includes a first (e.g., outer) shaft, a second (e.g., middle) shaft, a third (e.g., inner) shaft, a coring element including a blade or cutting edge, and a mesh. The coring element may comprise a multi-arm coring element, such as a coring element that is resilient or self-expanding and/or comprises a central region/portion/segment connected (e.g., integrally) to multiple arms, each of which may extend from the central segment. The coring element has a first, proximal end and a second, distal end. The first end of the coring element is coupled to the first shaft and the second end of the coring element is coupled to the second shaft. The mesh includes an open, proximal end and a second, distal end. Here, “open” in “open end” means that this end is the end of mesh into which clot material passes to enter the mesh. The open end of the mesh is secured to a proximal portion (distal to the proximal end) of the coring element and the second end of the mesh is coupled to the third shaft. Neither the second end nor any portion of the distal half of the coring element is secured to the mesh. The coring element does not have an opening bounded by uninterrupted coring element material. In some embodiments, the proximal portion of the coring element is secured to the open end of the mesh by a structure (e.g., suture material or wire material) that is separate from (i.e., not part of) the coring element and separate from the mesh. In some embodiments, most of (e.g., a majority of, including a majority of the length of) the coring element overlaps with the mesh. In some embodiments, most of (e.g., a majority of, including a majority of the length of) the coring element is positioned within the mesh (e.g., is surrounded by mesh material). In embodiments in which the coring element is self-expanding, such as because it is formed from self-expanding material or has been treated to have an unconstrained configuration that is at least somewhat resilient when constrained, the coring element may be referred to as a coring and biasing element because it is or has a structure that cores and because it is or has a structure that biases the open end of the mesh.

In another example, a system includes a shaft assembly including a plurality of shafts, a coring element coupled to the shaft assembly, and a mesh. The coring element includes a first portion and a second portion. The first portion includes a first pair of arms and a second pair of arms, and the first pair of arms includes a blade. The mesh is coupled to the shaft assembly and secured to the second pair of arms of the first portion of the coring element. The coring element may be resilient or self-expanding and/or comprises a central region/portion/segment connected (e.g., integrally) to the arms of the first and second pairs of arms.

In another example, an apparatus includes a coring element and a mesh. The coring element includes a first portion and a second portion. The first portion includes a first pair of arms and a second pair of arms, and the first pair of arms includes a blade. For example, each arm of the first pair of arms includes a blade portion. The mesh includes a plurality of end loops. The mesh is coupled to the second pair of arms of the first portion of the coring element by a wire that is wound around the plurality of end loops and the second pair of arms. The coring element may be resilient or self-expanding and/or comprises a central region/portion/segment connected (e.g., integrally) to the arms of the first and second pairs of arms.

As used herein, a resilient member is an elastic component that repeatedly stores and releases mechanical energy. For example, a resilient member may be any suitable spring (e.g., coil spring, extension/tension spring, machined spring, etc.).

The term “coupled” is defined as connected, although not necessarily directly, and not necessarily mechanically. The terms “a” and “an” are defined as one or more unless this disclosure explicitly requires otherwise. The term “end” refers to a concluding portion of the referenced structure and is not limited to the terminus of the referenced structure. Herein, translational movement, unless context indicates otherwise, is movement proximal or distal to the operator. The terms “substantially” and “approximately” are each defined as largely but not necessarily wholly what is specified—and include what is specified—as understood by a person of ordinary skill in the art. In any disclosed embodiment, the terms “substantially” and “approximately” may each be substituted with “within [a percentage] of” what is specified, where the percentage includes 0.1, 1, 5, and 10 percent.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

An apparatus or system, or a component of an apparatus or a system, that is configured in a certain way is configured in at least that way, but it can also be configured in other ways than those specifically described. Furthermore, reference in the appended claims to an apparatus or system or a component of an apparatus or system being adapted to, arranged to, capable of, configured to, or enabled to perform a particular function encompasses that apparatus, system, component, whether or not it or that particular function is activated, turned on, or unlocked, as long as that apparatus, system, or component is so adapted, arranged, capable, configured, or enabled.

Furthermore, all numerical ranges herein should be understood to include all integers, whole or fractions, within the range, inclusive of the ends of the ranges. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The feature or features of one embodiment may be applied to other embodiments, even though not described or illustrated, unless expressly prohibited by this disclosure or the nature of the embodiments.

Some details associated with the embodiments are described above and others are described below.

Additional features and advantages of the disclosed methods and apparatuses are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

A further understanding of the nature and advantages of the present disclosure may be realized by reference to the following drawings. In the appended figures, similar components or features may have the same reference label. Further, various components of the same type may be distinguished by following the reference label with a second label that distinguishes among the similar components. Like reference numbers and designations in the various drawings indicate like elements.

FIG. 1 is a perspective view of an example thrombectomy system, according to an embodiment of the present disclosure.

FIG. 2 is a box diagram of a funnel sheath-dilator system, according to an embodiment of the present disclosure.

FIG. 3 is a perspective view of an example implementation of the system of FIG. 1, according to an embodiment of the present disclosure.

FIG. 4 is an exploded view of the implementation of FIG. 3, according to an embodiment of the present disclosure.

FIGS. 5 to 8 are a series of exploded views of the introducer sheath, according to an embodiment of the present disclosure.

FIG. 9A is an exploded view of a hub of the introducer sheath, according to an embodiment of the present disclosure.

FIG. 9B is a cross-section of the introducer sheath, according to an embodiment of the present disclosure.

FIGS. 10A and 10B are each exploded views of a dilator, according to an embodiment of the present disclosure.

FIG. 10C is a perspective view of a distal end of an outer shaft of the dilator in relation to a cross section of a member of the dilator, according to an embodiment of the present disclosure.

FIGS. 10D and 10E are each cross sections of a proximal end of the dilator, according to an embodiment of the present disclosure.

FIGS. 11A and 11B are a pair of side views of the dilator showing a retraction of the cap, according to an embodiment of the present disclosure.

FIGS. 12A to 12D are a series of perspective views of a process of deploying a funnel of an introducer set, according to an embodiment of the present disclosure.

FIG. 12E is a perspective view of a portion of the system of FIG. 2 depicting aspiration being applied, according to an embodiment of the present disclosure.

FIG. 13 is a schematic cross-sectional view of part of a formation process of the cap and distal tip of the dilator, according to an embodiment of the present disclosure.

FIG. 14 is a flow chart of a method of introducing an introducer set into a patient, according to an embodiment of the present disclosure.

FIG. 15 is a block diagram of an extractor device, according to an embodiment of the present disclosure.

FIG. 16 is a perspective view of an example implementation of the extractor device, according to an embodiment of the present disclosure.

FIG. 17 is a perspective view of the extractor device with the extractor catheter removed, according to an embodiment of the present disclosure.

FIG. 18A is a perspective view of a handle of the extractor device with a portion of the housing of the handle removed, according to an embodiment of the present disclosure.

FIG. 18B is a perspective view of a portion of the housing, according to an embodiment of the present disclosure.

FIG. 19 is a perspective view of a shaft assembly of the handle, according to an embodiment of the present disclosure.

FIG. 20 is a perspective view of a sliding member of the handle, according to an embodiment of the present disclosure.

FIG. 21 is a perspective view of a middle shaft hub of the handle, according to an embodiment of the present disclosure.

FIG. 22 is a perspective view of an outer shaft hub of the handle, according to an embodiment of the present disclosure.

FIG. 23 is an exploded view of the middle shaft hub of the handle, according to an embodiment of the present disclosure.

FIG. 24 is an exploded view of the outer shaft hub of the handle, according to an embodiment of the present disclosure.

FIG. 25 is a cross-section (with the cross hatching omitted) perpendicular to the central axis of the shaft assembly of the middle shaft hub assembled with the components interior to the middle shaft hub shown for context, according to an embodiment of the present disclosure.

FIG. 26 is a cross-section (with the cross hatching omitted) perpendicular to the central axis of the shaft assembly of the outer shaft hub assembled with the components interior to the outer shaft hub shown for context, according to an embodiment of the present disclosure.

FIG. 27 is an upward-looking perspective view of a dial of the handle coupled to a pivot member of the handle, according to an embodiment of the present disclosure.

FIG. 28A is a perspective view of the dial, according to an embodiment of the present disclosure.

FIG. 28B is a perspective view of the dial, according to an embodiment of the present disclosure.

FIG. 29 is a perspective view of the pivot member, according to an embodiment of the present disclosure.

FIG. 30 is a perspective view of a trigger of the handle, according to an embodiment of the present disclosure.

FIG. 31 is a perspective view of a knob of the handle in relation to the housing of the handle, according to an embodiment of the present disclosure.

FIG. 32 is a cross-section of the handle, according to an embodiment of the present disclosure.

FIG. 33 is a magnified side view of the handle with a portion of the housing removed, according to an embodiment of the present disclosure.

FIG. 34A is an exploded view of a cover sleeve hub of the handle, according to an embodiment of the present disclosure.

FIG. 34B is a perspective view of the cover sleeve hub, according to an embodiment of the present disclosure.

FIG. 34C is a perspective view of the cover sleeve hub coupled to a clip of the sliding member of the handle, according to an embodiment of the present disclosure.

FIG. 35A is a cross section of the cover sleeve hub coupled to the clip of the sliding member, according to an embodiment of the present disclosure.

FIG. 35B is a cross-section along the central axis of the shaft assembly of the assembled middle shaft hub of the handle, according to an embodiment of the present disclosure.

FIG. 35C is a cross-section along the central axis of the shaft assembly of the assembled outer shaft hub, according to an embodiment of the present disclosure.

FIG. 36 is a side view of the assembled extractor catheter with a mesh of the extractor catheter shown in outline form perspective, according to an embodiment of the present disclosure.

FIG. 37 is a perspective view of the extractor catheter in isolation, according to an embodiment of the present disclosure.

FIG. 38 is an exploded view the extractor catheter, according to an embodiment of the present disclosure.

FIG. 39 is a top view of a coring element of the extractor catheter, according to an embodiment of the present disclosure.

FIG. 40 is a side view of the coring element, according to an embodiment of the present disclosure.

FIG. 41 is a perspective view showing a proximal end of the mesh, according to an embodiment of the present disclosure.

FIG. 42 is a perspective view showing the proximal end of the mesh coupled to the coring element, according to an embodiment of the present disclosure.

FIG. 43A is a perspective view of a distal end of the coring element relative to a cap for coupling the distal end to a shaft of the shaft assembly, according to an embodiment of the present disclosure.

FIG. 43B is a top view of the coring element coupled to shafts of the shaft assembly, according to an embodiment of the present disclosure.

FIG. 44 is a side view of the extractor device, according to an embodiment of the present disclosure.

FIG. 45 is a perspective view of the extractor device, according to an embodiment of the present disclosure.

FIGS. 46A to 46C are a series of schematic side views of the coring element coupled to the shaft assembly, according to an embodiment of the present disclosure.

FIG. 47 is a side view of the mesh having a length distal of the open end that has substantially the same shape perpendicular to an axis centered within the mesh, according to an embodiment of the present disclosure.

FIG. 48 is a side view of the mesh having a shape that tapers toward the distal end of the mesh, according to an embodiment of the present disclosure.

FIG. 49 is a side view of the mesh having a bump, according to an embodiment of the present disclosure.

FIG. 50 is a side view of the mesh having multiple bumps, according to an embodiment of the present disclosure.

FIGS. 51A to 51C are a series of top views of the extractor catheter with different mesh lengths, according to an embodiment of the present disclosure.

FIG. 52 is a flow chart of a method for extracting clot from a vascular system of a patient, according to an embodiment of the present disclosure.

### DETAILED DESCRIPTION

The detailed description set forth below, in connection with the appended drawings, is intended as a description of various configurations and is not intended to limit the scope of the disclosure to solely that described explicitly herein. Rather, the detailed description includes specific details for the purpose of providing a thorough understanding of the inventive subject matter. It will be apparent to those skilled in the art that these specific details are not required in every case and that, in some instances, well-known structures and components are shown in block diagram form for clarity of presentation.

A new and innovative vascular introducer set is provided that includes a dilator having a rotation mechanism for deploying a funnel of an introducer sheath. The vascular introducer set can be inserted into a vascular system of a patient, such as over a guidewire. The funnel is in a constrained state in which at least a portion thereof is within a cap of the dilator when the vascular introducer set is first inserted into the vascular system. Once the introducer sheath is seated at a desired location in a blood vessel of the vascular system, the funnel can be released using the rotation mechanism. To do so, a first component of the dilator can be rotated to cause the cap to advance away from the introducer sheath. When the cap no longer constrains the funnel, the funnel is released into an expanded (or unconstrained) state.

With the funnel in the expanded state, the dilator can then be removed from the blood vessel. To do so, the first component can be rotated in the opposite direction, which retracts the cap toward the introducer sheath. With the cap retracted, the dilator can be withdrawn out of the introducer sheath. At this point, the introducer sheath is set in place to aid in the introduction of a device into the vascular system of the patient. For example, a new and innovative extractor device provided herein can be introduced into the vascular system of the patient through the introducer sheath, including through the funnel, though other devices may be introduced.

The extractor device can be used for the removal of high-volume, long-length mixed clot that may have a wall adherent component. For example, the extractor device may be used for peripheral venous DVT clot removal. The extractor device includes a control handle having a 3-shaft assembly that includes an inner shaft, a middle shaft, and an outer shaft. While the outer shaft is fixed translationally (axially) to a housing of the control handle, the inner shaft and the middle shaft may translate relative to the housing based on an adjustment system of the control handle.

The extractor device further includes an extractor catheter coupled to the control handle. More specifically, the extractor catheter is coupled to the 3-shaft assembly, such that the extractor catheter may be manipulated based on the control handle. The extractor catheter includes a coring element and a mesh (e.g., a mesh element) coupled to the coring element. The coring element includes a blade or cutting edge configured to physically separate a blood clot from a blood vessel wall or break down a blood clot within a blood vessel. For instance, the extractor catheter is utilized to collect both the clot from the lumen of the vessel and clot that adheres to the vessel well. The separated or broken down clot can be collected, or captured, within the mesh. In at least some embodiments, a length of the mesh may be shortened using the control handle, which can allow for moving the extractor catheter closer to an IVC filter and/or easier cleaning of clot from the mesh.

### Thrombectomy System Embodiments

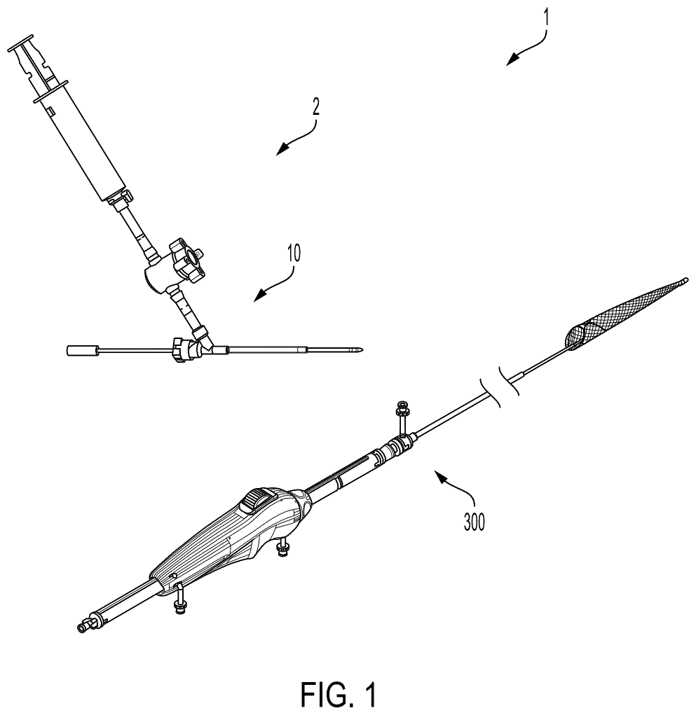

FIG. 1 illustrates a thrombectomy system 1 that may be used when performing a thrombectomy to remove a clot from a blood vessel of a patient. The thrombectomy system 1 generally includes a system 2 and an extractor device 300. The system 2 includes a vascular introducer set 10 that can be used to introduce the extractor device 300 into a vascular system of a patient. The system 2 may further include components that aid in clot removal, such as by providing aspiration, as will be discussed herein. The extractor device 300 can be used to separate a clot from a vessel wall and remove the clot from the patient. Examples of such clots include high-volume, long-length mixed clots with a wall-adherent component, such as those giving rise to peripheral venous DVT. For example, the clot may be separated from the vessel wall using, and collected in, a collapsible mesh of the extractor device 300. While the system 2 and the extractor device 300 are described together as the thrombectomy system 1, the system 2 may be utilized with other suitable devices, and the extractor device 300 may be introduced into a vascular system of a patient using other suitable introducer sets. Stated differently, the utility of the system 2 is separate from the utility of the extractor device 300.

### Introducer Set Embodiments

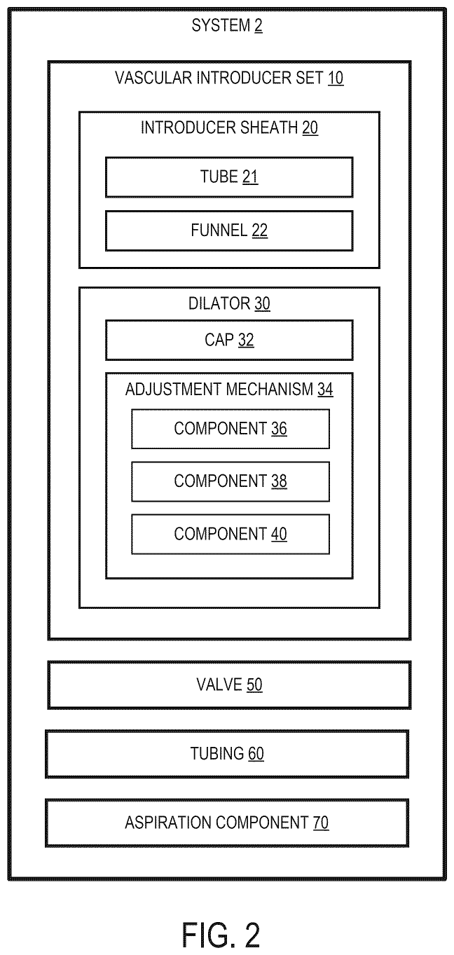

FIG. 2 is a box diagram of the system 2 that includes the vascular introducer set 10. The vascular introducer set 10 generally includes an introducer sheath 20 and a dilator 30. The introducer sheath 20 includes at least a tube 21 and a funnel 22. The funnel 22 is coupled to a distal portion of the tube 21. The dilator 30 includes at least a cap 32 and an adjustment system 34. The adjustment system 34 is adapted to advance, or retract, the cap 32. For example, the adjustment system 34 includes at least components 36, 38, and 40 that operate together to advance or retract the cap 32 according to physician input. For example, rotating the component 36 in a first direction may cause the component 40, which is coupled to the cap 32, to translate relative to the component 38 such that the cap 32 is advanced away from the component 38. In this example, rotating the component 36 in a second, opposing direction may cause the component 40, and therefore the cap 32, to translate relative to the component 38 such that the cap 32 is retracted toward the component 38. In various embodiments, the system 2 may also include one or more of a valve 50 (e.g., stopcock valve), tubing 60, or an aspiration component 70, which will be described below.

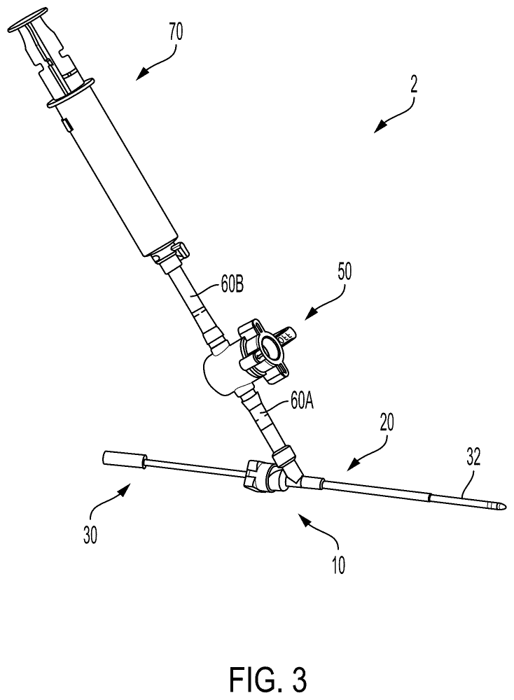

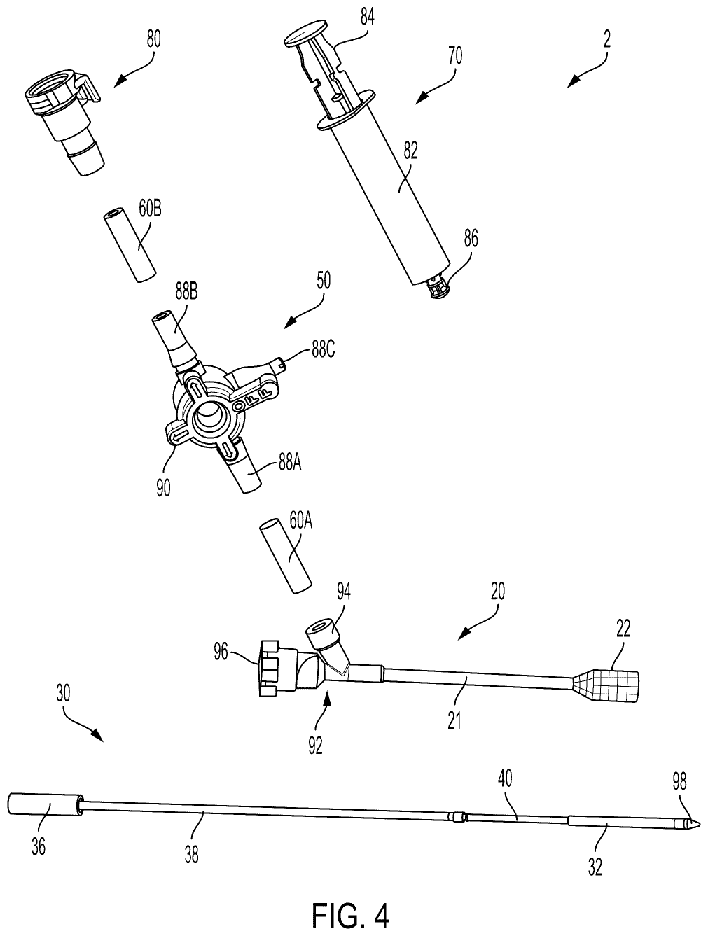

FIGS. 3 and 4 show an example implementation of the system 2. In the perspective view of FIG. 3, the vascular introducer set 10 is shown in a pre-deployment, as-manufactured state with the dilator 30 positioned through the introducer sheath 20. With the vascular introducer set 10 in the pre-deployment state, the funnel 22 of the introducer sheath 20 is maintained in a constrained state because the funnel 22 is at least partially within the cap 32; in the depicted embodiment, the funnel 22 is (completely) within the cap 32 and is therefore not visible in FIG. 3. Conversely, when no portion of the funnel 22 is constrained by the cap 32, as in FIG. 4, the funnel 22 transitions to an expanded state. In this way, the funnel 22 is self-expanding. The funnel 22 can be a woven mesh, without a coating or jacket, including mesh openings through which fluid can flow. The funnel 22 can be sized such that a distal portion 103 of the funnel 22 contacts a blood vessel wall when released to the expanded state within a blood vessel. In other embodiments, the funnel 22 may be sized such that the distal portion 103 does not contact the blood vessel wall. A proximal portion 102 of the funnel 22 is coupled to the tube 21 of the introducer sheath 20, such as via a heat-shrink polymer sleeve. For example, the heat-shrink polymer sleeve is disposed around the proximal portion 102 and heating the heat-shrink polymer sleeve causes the sleeve to shrink, thereby coupling the proximal portion 102 to the tube 21. A proximal end of the tube 21 is coupled to a hub 92 of the introducer sheath 20.

The hub 92 may include a port 94 (which may be characterized as a side port) that can be used to couple the introducer sheath 20 to another component. For instance, in the illustrated example, the port 94 is coupled to a port 88A of the valve 50 (which may be characterized as a three-way valve, a stopcock, or a three way stopcock) via a first portion of tubing 60A. The valve 50 may include various other ports, such as ports 88B and 88C, for fluidly coupling components to the introducer sheath 20. For instance, in the illustrated example, the aspiration component 70 is coupled to the port 88B of the valve 50 via a second portion of tubing 60B and a connector 80, which can be a quick-release connector that enables rapid coupling and decoupling of the aspiration component 70 from the introducer sheath 20. The aspiration component 70 is a component capable of generating a suctioning force. For example, the aspiration component 70 is illustrated as a conventional manual syringe including a body 82, a plunger 84, and a connecting portion (or adapter) 86 that can be coupled to a tip (not shown) of the body 82 on one end and connectable via its other end to the connector 80. The syringe may be lockable, and take the form of a VACLOCK® syringe (by Merit Medical Systems), where the body 82 (or barrel) includes a locking component (not shown) that extends radially inward and the plunger 84 includes a notch that can be rotated to avoid the locking component as well as fins (not shown) that can be rotated to interfere with the locking component at a desired level of suction, thereby locking the position of the plunger relative to the position of the barrel. The aspiration component 70 may, however, be implemented as other suitable instrumentation in other examples. The valve 50 includes a switch 90 for controlling which of the ports 88A, 88B, 88C are in fluid communication with one another.

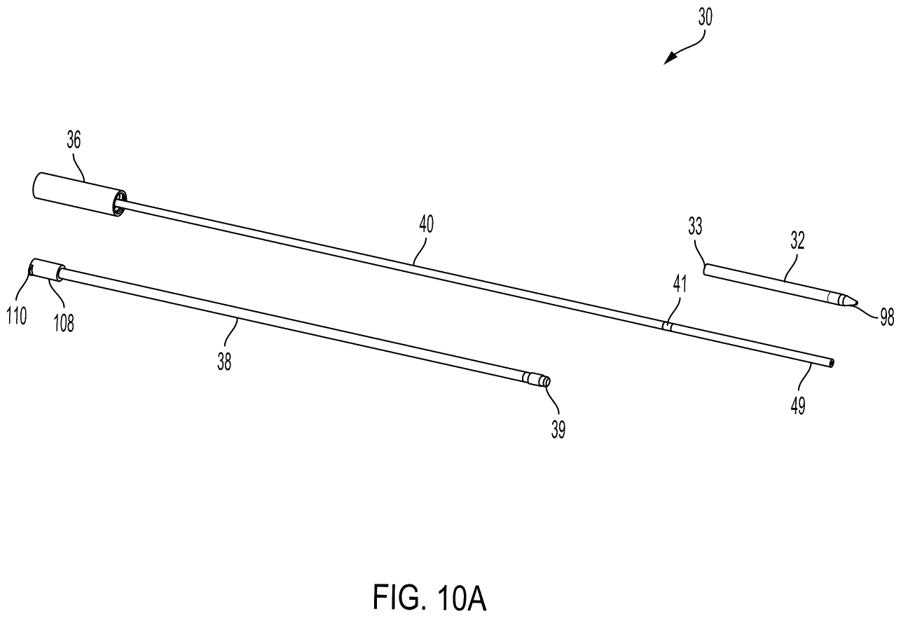

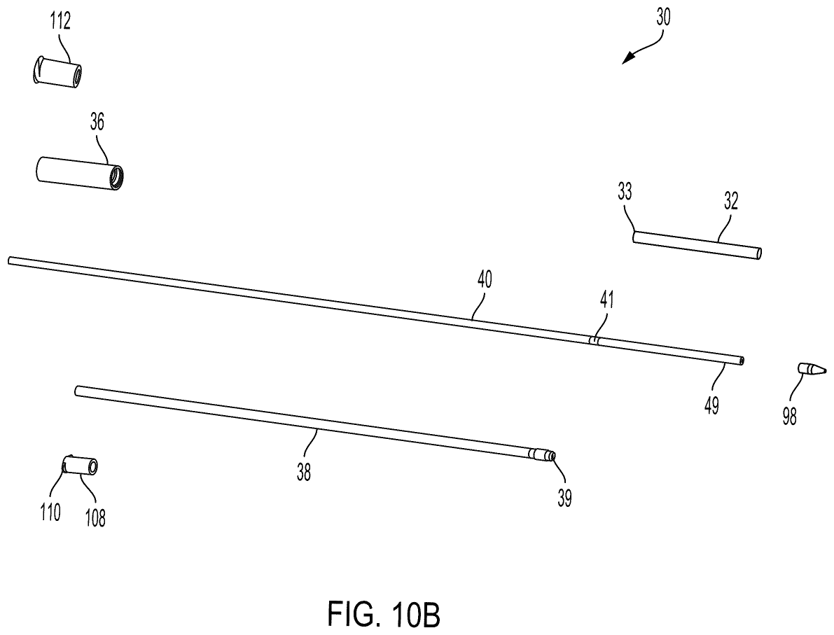

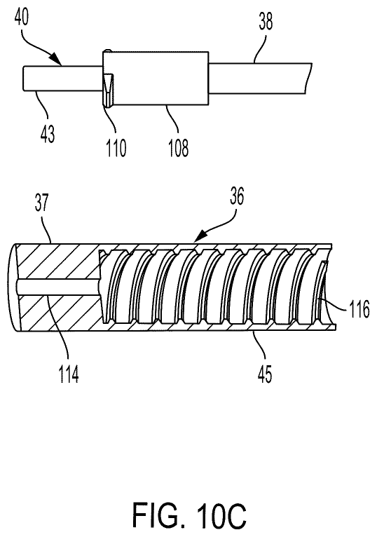

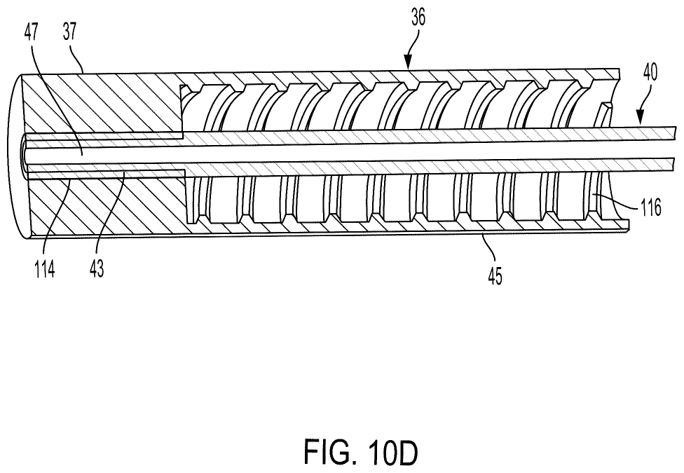

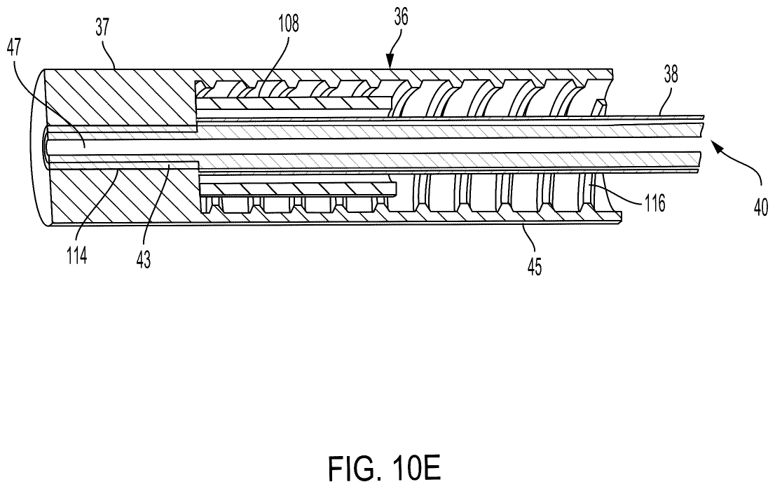

A proximal end of the hub 92 includes an opening 96 for receiving the dilator 30. The hub 92 may also include a mechanism, such as a compression gasket, for clamping onto the component 38 of the dilator 30 to create a seal around the component 38 and limit translational and rotational movement of the component 38 relative to the hub 92. In the illustrated example, the component 38 is implemented as an outer shaft (e.g., a tube) and the component 40 is implemented as an inner shaft (e.g., a tube) disposed through the outer shaft 38. The inner shaft 40 includes a channel (or lumen) 47 (FIG. 10D) extending through the inner shaft 40. The channel 47 may accept, for example, a guidewire as the inner shaft 40 is advanced (distally) or retracted (proximally) over, or along, the guidewire. The component 36 is an adjusting member implemented in the illustrated example as a rotatable barrel that is coupled to a proximal end of the inner shaft 40. The cap 32 is coupled to a distal end of the inner shaft 40. The cap 32 includes a distal tip 98 having a tapered end that reduces the risk of trauma when introducing the dilator 30 into a blood vessel. The distal tip 98 may be characterized as an atraumatic distal tip.

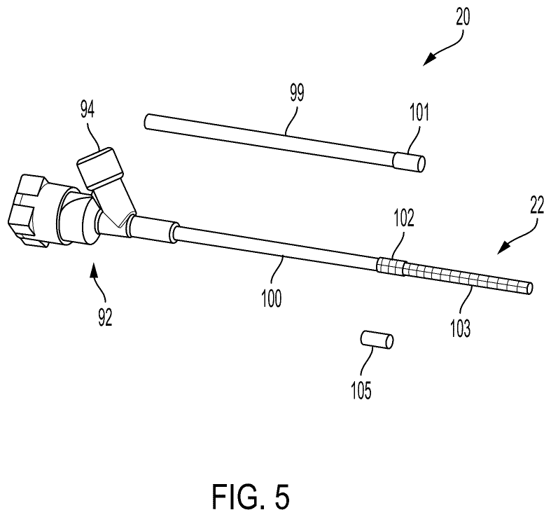

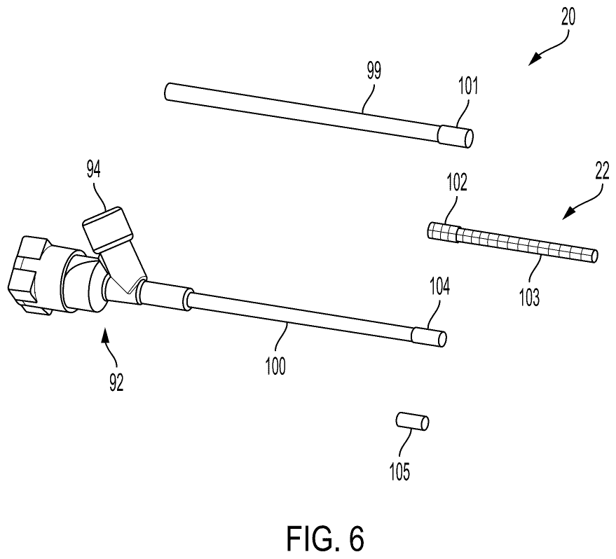

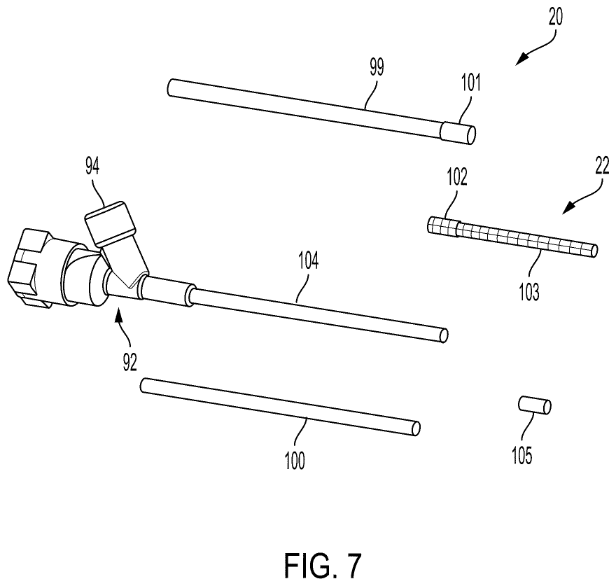

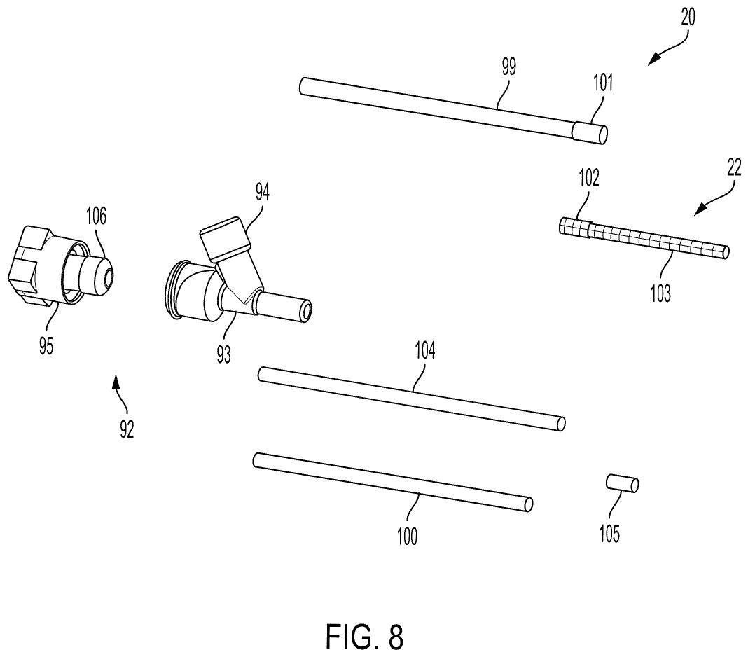

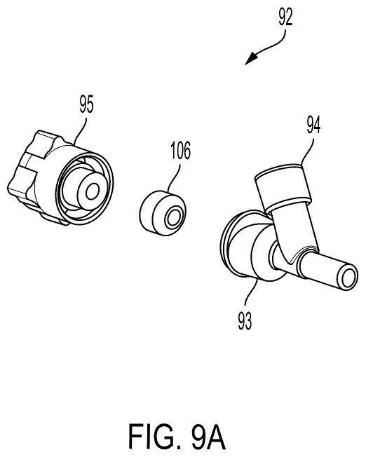

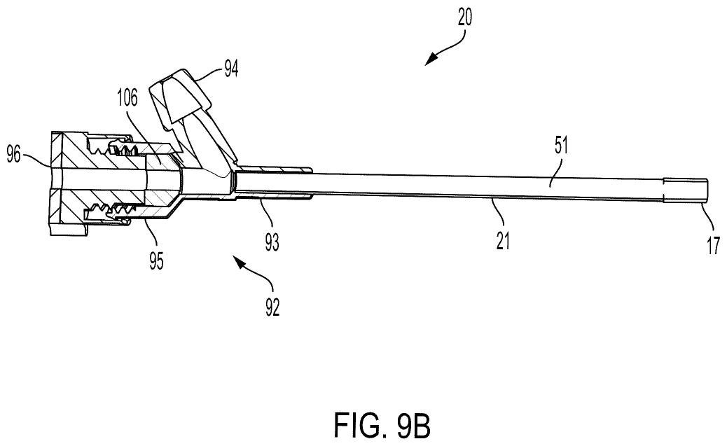

FIGS. 5 to 9B illustrate an example structure of the introducer sheath 20 in further detail. The tube 21 of the introducer sheath 20 defines a lumen 51 (FIG. 9B) and may include various components. For example, the tube 21 may include an outer jacket 99, a reinforcement member 100, and an inner liner 104, as shown in the illustrated example. The reinforcement member 100 is disposed around the inner liner 104 and may stiffen the tube 21 to allow for greater directional control. In various embodiments, the reinforcement member 100 may be a reinforced coil or braid. The outer jacket 99 is disposed around the reinforcement member 100. The reinforcement member 100 may be laminated between the outer jacket 99 and the inner liner 104.

In various embodiments, the material of the outer jacket 99 may include a polymer, such as an elastomer, and more particularly such as a thermoplastic elastomer. For example, the outer jacket 99 may include polyether block amide (PEBA) (e.g., PEBAX®), PEBA with a slip additive (e.g., EverGlide®), or Nylon 12. In various embodiments, the material of the reinforcement member 100 may include a metal, such as steel, and more particularly such as stainless steel. The inner liner 104 is configured to provide a low friction surface for a device or clot to pass through. For example, in various embodiments, the inner liner 104 may include a polymer, such as an elastomer, and more particularly such as a thermoplastic elastomer. For example, the inner liner 104 may include polytetrafluoroethylene (PTFE), polyether block amide (PEBA) (e.g., PEBAX®), or PEBA with a slip additive (e.g., EverGlide®).

In various embodiments, the outer jacket 99 may include a radiopaque marker 101. The radiopaque marker 101 may, for example, include bismuth oxychloride, tungsten, or another suitable radiopaque material.

A proximal portion 102 of the funnel 22 may be coupled to the inner liner 104 in some embodiments. In some embodiments, the distal portion 103 of the funnel 22 may taper into the proximal portion 102, which can enable better bonding between the proximal portion 102 and the inner liner 104 or another component of the tube 21. The outer jacket 99 is disposed around the proximal portion 102 of the funnel 22. It should be appreciated that the tube 21 is not limited to this structure and may have other suitable structures for the intended purpose of the tube 21. For example, the proximal portion 102 of the funnel 22 may be coupled to a different component of the tube 21.

In various embodiments, the hub 92 may include a first component 93 and a second component 95. The first component 93 includes the port 94. The second component 95 may be coupled to the first component 93. For example, the first component 93 may include first threading (e.g., male threading, which may take the form of a continuous (single) male thread) and the second component 95 may include second threading (e.g., female threading, which may take the form of a continuous (single) female thread) that may be engaged with one another. A compression seal 106 may be positioned between the first component 93 and the second component 95. When the dilator 30 is positioned through the hub 92, and the second component 95 is tightened relative to the first component 93, the compression seal 106 is compressed and locks the outer shaft 38 of the dilator 30 in place relative to the hub 92 and creates a seal (e.g., a fluid-tight seal) around the outer shaft 38. It should be appreciated that the hub 92 is not limited to this structure and may have other suitable structures for the intended purpose of the hub 92.

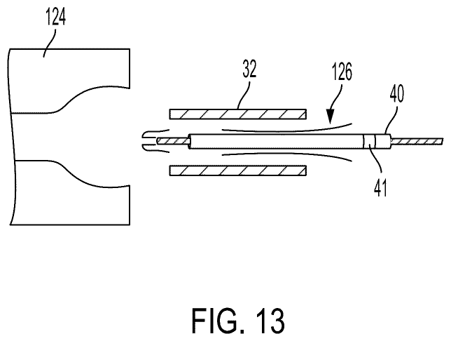

FIGS. 10A to 10E illustrate an example structure of the adjustment system 34 of the dilator 30 in further detail. As described, the inner shaft 40 is coupled to the rotatable barrel 36. For example, as best seen in FIGS. 10C and 10D, a proximal end 43 of the inner shaft 40 may be coupled to a first portion 37 of the rotatable barrel 36 that includes a channel 114 through which the proximal end 43 is disposed. The proximal end 43 may be coupled to the first portion 37 by adhesive or another suitable coupling method. A second portion 45 of the rotatable barrel 36 includes interior threading 116 (e.g., female threading, which may take the form of a continuous (single) female thread). A distal end 49 of the inner shaft 40 is coupled to the cap 32. The inner shaft 40 includes a radiopaque marker 41, such as a tungsten extrusion, which may be bonded to the inner shaft 40 and serve as a location aid during a fluoroscopic procedure. The radiopaque marker 41 is disposed on the inner shaft 40 at the position of the proximal end 33 of the cap 32, which can make forming the cap 32 and distal tip 98 on the inner shaft 40 easier. For example, FIG. 13 is a schematic cross-sectional view of part of a formation process of the cap 32 and distal tip of the dilator. An extruded cap 32 may be added on to the inner shaft 40 using a tipping process that forms the distal tip (e.g., 98, shown in FIG. 10A) in the same process. The tipping process involves using a mandrel 126 and a mold 124. By disposing the radiopaque marker 41 at the location where the proximal end of the cap 32 will be, the radiopaque marker 41 does not interfere with the formation process. In some embodiments, however, the radiopaque marker 41 may be disposed at other suitable locations on the inner shaft 40. In some embodiments, a formation process may include bonding a molded distal tip (e.g., 98, shown in FIG. 10A) to an extruded cap 32. In some embodiments, the proximal end of the cap 32 can include the radiopaque marker 41 instead of, or in addition to, the inner shaft 40. In some embodiments, the radiopaque marker 41 may be omitted.

Returning to FIGS. 10A to 10E, the outer shaft 38 includes a coupling member 108. The coupling member 108 may be coupled to, or integral with, the tube portion of the outer shaft 38. At least one thread 110 (e.g., at least one exterior, male thread) is disposed on the coupling member 108. The at least one thread 110 can be engaged with the threading 116 of the rotatable barrel 36. In this way, rotating the rotatable barrel 36 relative to the outer shaft 38 causes the at least one thread 110 to travel within the threading 116 such that the rotatable barrel 36 translates relative to the outer shaft 38. In at least some embodiments, the outer shaft 38 includes a tapered distal end 39. The tapered distal end 39 may receive the proximal end 33 of the cap 32 when the cap 32 is fully retracted, which reduces or eliminates a gap between the outer shaft 38 and the cap 32.

In some embodiments, various types of a Luer activated valve 112 may be included at a proximal end of the rotatable barrel 36. In such embodiments, the Luer activated valve 112 can be used as a flush port.

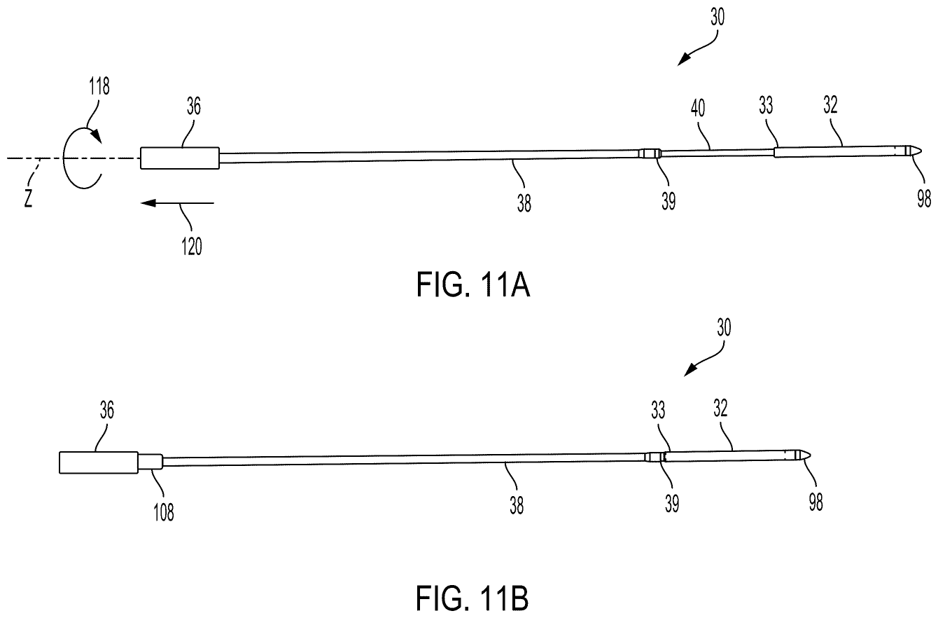

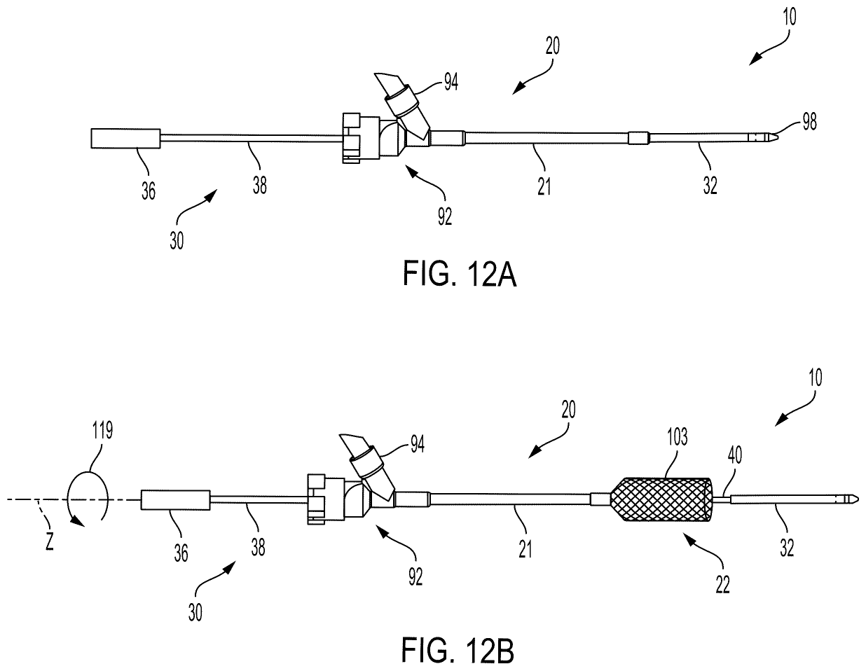

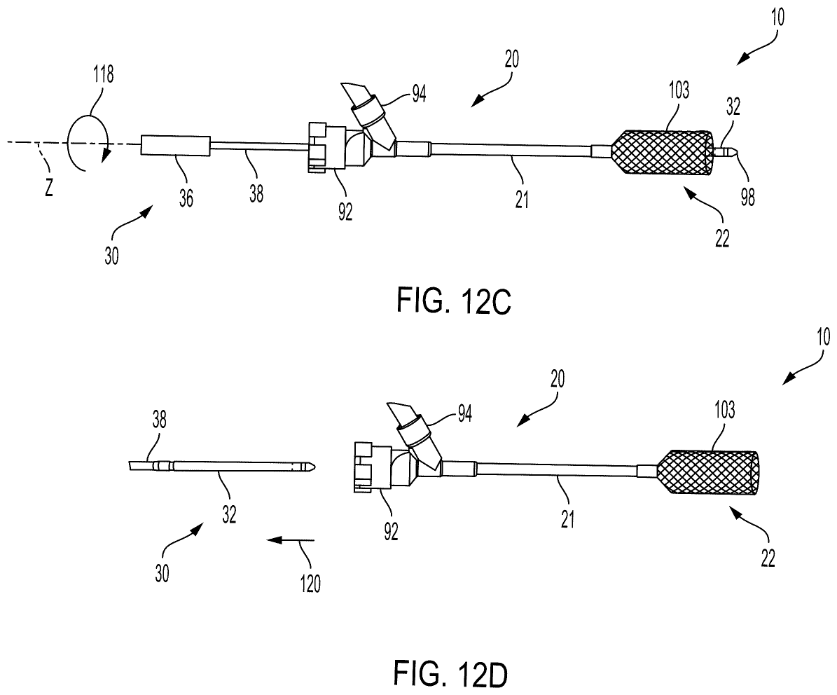

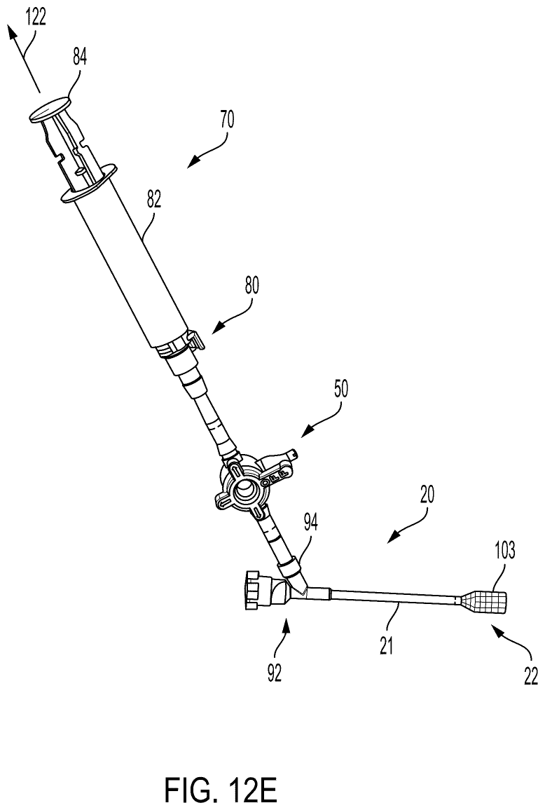

FIGS. 11A and 11B illustrate a retraction operation of the adjustment system 34 with the above-described structure. In FIG. 11A, the cap 32 is advanced away from (i.e., moved distally relative to) the tapered distal end 39 of the outer shaft 38. From this position, considering the outer shaft 38 to be restricted from movement, rotating the rotatable barrel 36 in the direction of arrow 118 about an axis Z retracts the cap 32 toward (i.e., moves the cap 32 proximally relative to) the tapered distal end 39. The axis Z extends through a length of (and is centered within) each of the rotatable barrel 36, the outer shaft 38, the inner shaft 40, and the cap 32. For instance, rotating the rotatable barrel 36 about the axis Z in the direction of the arrow 118 causes the at least one thread 110 of the coupling member 108 to advance through the threading 116 of the rotatable barrel 36, which causes the rotatable barrel 36 to translate relative to the outer shaft 38 in the direction of the arrow 120. Because the cap 32 is coupled to the inner shaft 40, and the inner shaft 40 is coupled to the rotatable barrel 36, the cap 32 translates in the direction of the arrow 120 along with the rotatable barrel 36. In some embodiments, the cap 32 can be retracted until the proximal end 33 of the cap 32 abuts the tapered distal end 39 as shown in FIG. 11B. It will be appreciated that rotating the rotatable barrel 36 about the axis Z in the direction opposite to the arrow 118 (e.g., the arrow 119 in FIG. 12B) causes the cap 32, and therefore the inner shaft 40 and the cap 32, to translate in the direction opposite the arrow 120 so as to advance the cap 32.

While the rotatable barrel 36 and the cap 32 in the illustrated example are configured such that rotating the rotatable barrel 36 clockwise advances the cap 32 and rotating the rotatable barrel 36 counter clockwise retracts the cap 32, it should be appreciated that the relevant threadings may be adjusted such that these directions may be reversed and, thus, rotating the rotatable barrel 36 clockwise will retract the cap 32, and rotating the rotatable barrel 36 counterclockwise will advance the cap 32. Additionally, while the adjustment system 34 is illustrated as including the at least one exterior thread 110 of the coupling member 108 and the interior threading 116 of the rotatable barrel 36, the coupling member 108 and the rotatable barrel 36 may have different suitable structures that enable advancing and retracting the inner shaft 40 relative to the outer shaft 38.

FIGS. 12A to 12E show a series of operations for deploying the funnel 22 of the vascular introducer set 10. In FIG. 12A, the second component 95 of the hub 92 is tightened to the first component 93 such that the compression seal 106 locks the outer shaft 38 of the dilator 30 in place relative to the hub 92 such that the outer shaft 38 is restricted from rotating or translating relative to the hub 92. The vascular introducer set 10 as shown in FIG. 12A is in a pre-deployment state. After an introducer is used to create an entry point into a vascular system of a patient (e.g., into a blood vessel), the vascular introducer set 10 as shown in FIG. 12A can be partially inserted into the blood vessel while a working portion of the vascular introducer set 10 remains outside of the patient. For example, beginning with the distal tip 98, the vascular introducer set 10 is advanced into the blood vessel until a first portion of the tube 21 of the introducer sheath 20 is inserted into the blood vessel. Stated differently, the hub 92 is not inserted into the blood vessel and, in some embodiments, a second portion of the tube 21 is not inserted into the blood vessel. In some embodiments, the vascular introducer set 10 may be inserted over a guidewire (not shown) inserted through the introducer providing the initial access to the interior of the blood vessel and that has been positioned as desired by the operator. In such embodiments, the vascular introducer set 10 is advanced over the guidewire such that the guidewire is positioned through the distal tip 98, cap 32, and inner shaft 40.

At this point, the funnel 22 is in the constrained state and at least partially (and, in the depicted embodiment, completely) within the cap 32. Once the introducer sheath 20 is positioned, or seated, at the desired location in the blood vessel, the funnel 22 can be released using the adjustment system 34. To do so, the rotatable barrel 36 is rotated about the axis Z in the direction of the arrow 119, which advances the cap 32 distally, away from the tube 21. When the cap 32 no longer constrains the funnel 22, as shown in FIG. 12B, the funnel 22 is released into the expanded state. The vascular introducer set 10 as shown in FIG. 12B is in a deployed state.

With the funnel 22 in the expanded state, the dilator 30 can then be removed from the vascular system. In some embodiments, as shown in FIG. 12C, the rotatable barrel 36 may be rotated about the axis Z in the direction of the arrow 118, which retracts the cap 32 proximally, toward the tapered distal end 39 of the outer shaft 38. For example, the cap 32 can be retracted until the proximal end 33 of the cap 32 is within the tube 21 or until the proximal end 33 abuts the tapered distal end 39 of the outer shaft 38. Retracting the cap 32 in this way, prior to withdrawing the dilator 30 from the introducer sheath 20, closes the gap between the cap 32 and the tapered distal end 39, which can reduce hang up when pulling the dilator 30 out through the introducer sheath 20. For instance, when the second component 95 of the hub 92 is loosened from the first component 93 so that the compression seal 106 releases the outer shaft 38 of the dilator 30 and the dilator 30 can be pulled out through the introducer sheath 20, the dilator 30 is no longer held centered axially relative to the funnel 22. Retracting the cap 32 while the dilator 30 remains centered axially, prior to loosening the second component 95, reduces the likelihood that the edge of the proximal end 33 of the cap 32 gets caught on the funnel 22 or another component of the introducer sheath 20 during withdrawal of the dilator 30, when the dilator 30 is no longer held centered axially. With the cap 32 retracted, the dilator 30 can be withdrawn out of the patient and out of the introducer sheath 20 in the direction of the arrow 120, as shown in FIG. 12D. In some embodiments, the cap 32 is not retracted, or is not retracted all the way to abut the tapered distal end 39 of the outer shaft 38, prior to withdrawing the dilator 30 from the patient and the introducer sheath 20.

At this point, the introducer sheath 20 is set in place to aid in the introduction of a device into the vascular system of the patient. For example, the extractor device 300 can be introduced into the vascular system of the patient through the introducer sheath 20, including through the funnel 22. At some point in a procedure that involves the extractor device 300, aspiration may need to be applied to suction a blood clot from the vascular system. For example, aspiration may be applied subsequent to the extractor device 300 being withdrawn out of the vascular system and the introducer sheath 20 with a captured blood clot. The aspiration may be applied to suction out bits of blood clot that were broken up by the extractor device 300 but remain in the vascular system. FIG. 12E shows the aspiration component 70 coupled to the hub 92 via the valve 50. The plunger 84 of the aspiration component 70 may be withdrawn in the direction of the arrow 122 to apply suction for aspirating the bits of blood clot. Other components may be coupled to the valve 50 as needed for the procedure.

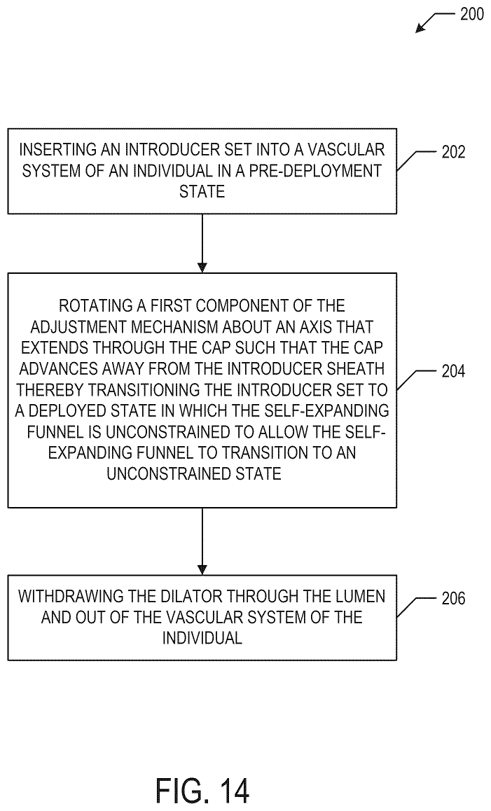

FIG. 14 shows a flow chart of an example method 200. The method 200 may be used for positioning an introducer sheath in a vascular system of a patient. At block 202, the method 200 includes inserting an introducer set (e.g., vascular introducer set 10) in a pre-deployment state into a vascular system of a patient. The vascular introducer set 10 includes an introducer sheath (e.g., introducer sheath 20) and a dilator (e.g., dilator 30). The introducer sheath 20 includes a tube (e.g., tube 21) defining a lumen (e.g., lumen 51), and a self-expanding funnel (e.g., funnel 22) coupled to the tube 21. The dilator 30 is disposed through the lumen 51 and includes a cap (e.g., cap 32) and an adjustment system (e.g., adjustment system 34). At least a portion of the self-expanding funnel 22 is disposed within the cap 32 in the pre-deployment state such that the self-expanding funnel 22 is maintained in a constrained state by the cap 32. The adjustment system 34 is configured to advance the cap 32 away from the tube 21.

At block 204, a first component (e.g., rotatable barrel 36) of the adjustment system 34 is rotated about an axis (e.g., axis Z) that extends through the cap 32 such that the cap 32 advances away from the introducer sheath 20 thereby transitioning the vascular introducer set 10 to a deployed state in which the self-expanding funnel 22 is unconstrained to allow the self-expanding funnel 22 to transition to an expanded state. In some embodiments, the first component 36 is coupled to a proximal end (e.g., proximal end 43) of a second component (e.g., inner shaft 40) of the adjustment system 34. In such embodiments, the cap 32 is coupled to a distal end (e.g., distal end 49) of the second component 40, and the second component 40 is disposed through a third component (e.g., outer shaft 38) of the adjustment system 34. In such embodiments, the first component 36 includes at least one first thread (e.g., threading 116) and the third component 38 includes at least one second thread (e.g., at least one thread 110), and rotating the first component 36 so as to advance the at least one first thread 116 relative to the at least one second thread 110 advances the cap 32 away from the introducer sheath 20.

At block 206, the dilator 30 is withdrawn through the lumen 51 and out of the vascular system of the patient. In some embodiments, the method 200 further includes rotating the first component 36 about the axis such that the cap 32 is retracted toward the tube 21 prior to withdrawing the dilator 30 through the lumen 51. In some embodiments, the cap 32 may be retracted such that a proximal end (e.g., proximal end 33) of the cap 32 abuts the tube 21, or enters the tube 21, prior to withdrawing the dilator 30 through the lumen 51. In some embodiments, the cap 32 may be retracted such that a proximal end (e.g., proximal end 33) of the cap 32 abuts a distal end (e.g., tapered distal end 39) of the third component 38 prior to withdrawing the dilator 30 through the lumen 51.

Example materials of the introducer sheath 20 and the dilator 30 will now be discussed. In various embodiments, the funnel 22 may include a shape-memory material, such as nitinol.

In various embodiments, the outer shaft 38 may include a polymeric material, such as an elastomer, and more particularly such as a thermoplastic elastomer. For example, the outer shaft 38 may include polyether block amide. In another example, the outer shaft 38 may include polyimide. In some embodiments, the outer shaft 38 may include (e.g., 20% by weight) barium sulfate for radiopacity. In some embodiments, the outer shaft 38 may include a lubrication additive to reduce friction between the outer shaft 38 and the inner shaft 40 while the inner shaft 40 slides through the outer shaft 38.

In various embodiments, the inner shaft 40 may include a polymeric material, such as an elastomer, and more particularly such as a thermoplastic elastomer. For example, the inner shaft 40 may include polyetheretherketone (PEEK). The inner shaft 40 provides a sufficient flexibility and stiffness to enter the body of a patient with sixty degrees of bending.

In various embodiments, the cap 32, including the distal tip 98, may include a polymeric material. For example, the cap 32 and the distal tip 98 may each include a Nylon polymer. In some embodiments, the distal tip 98 may be a different material from the remaining portion of the cap 32.

Example dimensions of the introducer sheath 20 and the dilator 30 will now be discussed. The rotatable barrel 36 may have an outer diameter within a range of 1 centimeter (cm) to 1.4 cm and/or may have a length of 8 cm. The outer shaft 38 may have an outer diameter within a range of 0.369 cm to 0.510 cm and/or may have a length of 31 cm. The inner shaft 40 may have an outer diameter within a range of 0.226 cm to 0.237 cm and/or may have a length of 39 cm. The cap 32 may have an outer diameter within a range of 0.369 cm to 0.510 cm and/or may have a length of 4 cm. The distal tip 98 may have a length of 5 millimeters (mm). The outer jacket 99 may have an outer diameter within a range of 0.474 cm to 0.640 cm. The inner liner 104 may have an outer diameter within a range of 0.409 cm to 0.549 cm. The funnel 22 may have a maximum outer diameter within a range of 12 mm to 14 mm, which enables the funnel 22 to be used in a vessel size (e.g., 6 mm diameter) that ensures there's adequate radial force between the funnel 22 and the vessel to prevent a leak to the proximal side of the funnel 22 or under the entry point.

### Extractor Device Embodiments

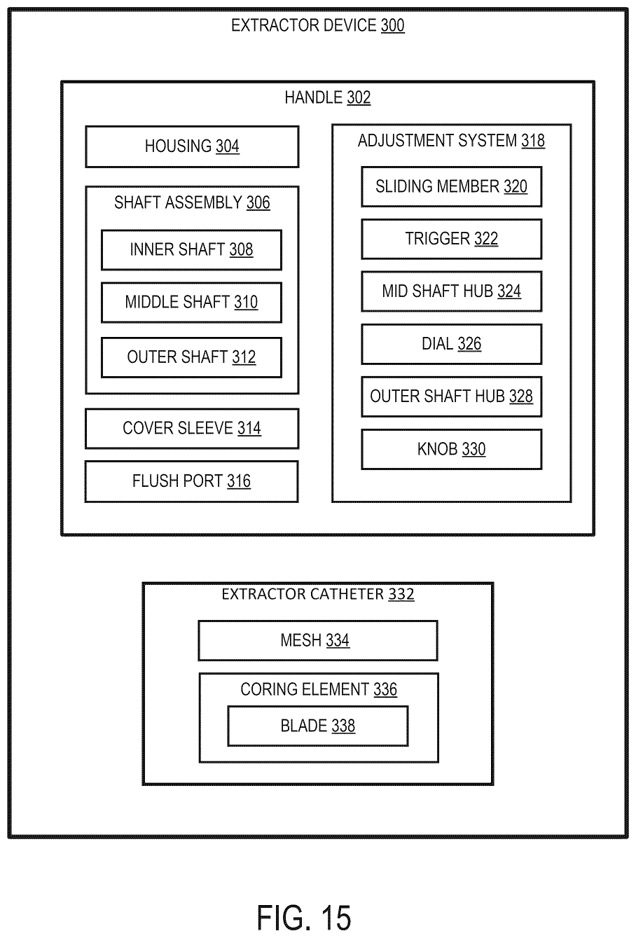

FIG. 15 is a block diagram of the extractor device 300. The extractor device 300 includes a handle 302 and an extractor catheter 332 coupled to the handle 302. The handle 302 may be used to manipulate the extractor catheter 332 or to allow the extractor catheter 332 to adjust freely. The handle 302 includes a housing 304 and a shaft assembly 306 that is partially disposed within the housing 304. The shaft assembly 306 is a 3-shaft system that includes an inner shaft 308, a middle shaft 310, and an outer shaft 312 that are concentrically arranged to create a telescoping effect such that the outer shaft 312 is disposed around a portion of the middle shaft 310 and the middle shaft 310 is disposed around a portion of the inner shaft 308. The handle 302 may also include an advanceable and retractable cover sleeve 314 disposed around a portion of the outer shaft 312. In some embodiments, the handle 302 includes at least one flush port 316 configured to enabling flushing of space between any two of the inner shaft 308, the middle shaft 310, the outer shaft 312, and the cover sleeve 314, or within the inner shaft 308.

The extractor catheter 332 includes a mesh 334 (e.g., a mesh element or a clot collecting element) and a coring element 336. The mesh 334 is coupled to the coring element 336. The coring element 336 includes a blade 338 configured to physically separate a blood clot from a vessel wall or break down a blood clot within a vessel. For instance, the extractor catheter 332 collects both clot material from the vessel's lumen (e.g., its center) and clot material that is being separated from the vessel's wall.

The handle 302 further includes an adjustment system 318 capable of adjusting, or allowing adjustment of, the extractor catheter 332 by adjusting, or allowing adjustment of, the relative positions between the inner shaft 308, the middle shaft 310, and the outer shaft 312. For example, a physician may operate the adjustment system 318 to manually manipulate the relative positions between the shafts 308, 310, and 312 or to allow the extractor catheter 332 to manipulate the relative positions between the shafts 308, 310, and 312. For example, blood vessels within the vasculature of a patient have different sizes and the adjustment system 318 may be operated to allow the extractor catheter 332 traveling through a blood vessel to conform to the blood vessel as the blood vessel narrows or widens, due in at least some embodiments to the self-expanding nature of the mesh and the coring element.

The adjustment system 318 includes a sliding member 320 coupled to the inner shaft 308, a middle shaft hub 324 coupled to the middle shaft 310, and an outer shaft hub 328 coupled to the outer shaft 312. The sliding member 320 includes a control portion 321 that a physician may grip and manually translate to control translation of the inner shaft 308. The adjustment system 318 further includes a trigger 322 for allowing or inhibiting movement of the sliding member 320, and a dial 326 for allowing, controlling, or inhibiting movement of the middle shaft hub 324. In some embodiments, a knob 330 is included with the adjustment system 318 that enables a physician to rotate, as a whole, the shaft assembly 306 and the sliding member 320.

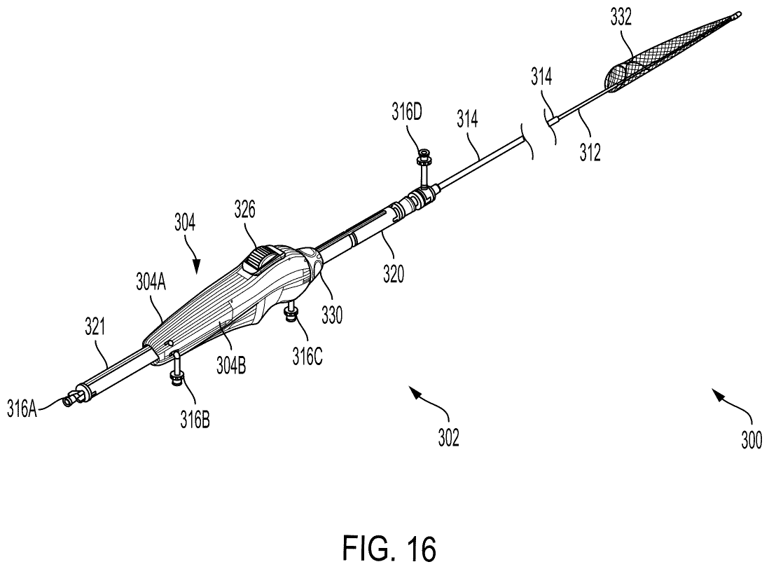

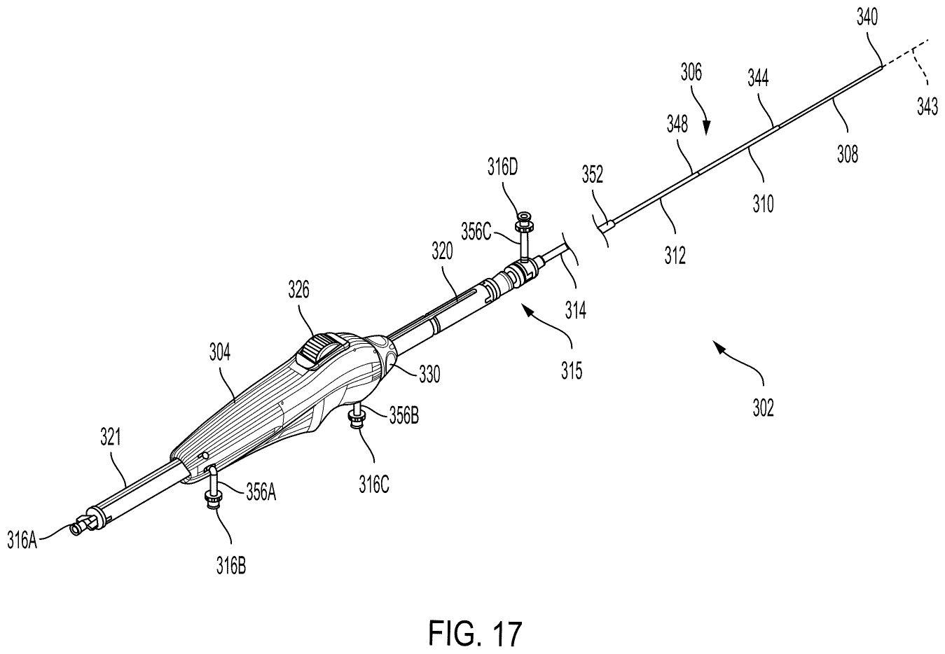

An example implementation of the extractor device 300 is shown in FIG. 16 and also in FIG. 17 with the extractor catheter 332 removed to allow the external portions of the shaft assembly 306 to be viewed. The handle 302 and its components shown in FIGS. 16 to 35C are drawn to scale, meaning the sizes of the depicted components are accurate relative to each other for at least the depicted embodiments of the handle; but as a person of ordinary skill in the art will understand, the present disclosure is not limited to the depicted embodiment of the handle, and other embodiments that do not possess the depicted sizes also fall within the scope of the relevant claims. As described, a portion of the inner shaft 308 is disposed within the middle shaft 310, and a portion of the middle shaft 310 is disposed within the outer shaft 312. A portion of the outer shaft 312 may be disposed within the cover sleeve 314. An axis 343 extends through a center of each of the inner shaft 308, the middle shaft 310, the outer shaft 312, and the cover sleeve 314.

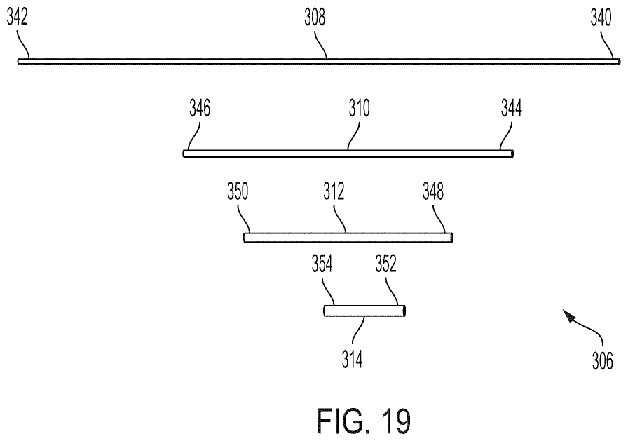

With reference to FIG. 17 and FIG. 19, the inner shaft 308 includes a distal end 340 and a proximal end 342, the middle shaft 310 includes a distal end 344 and a proximal end 346, and the outer shaft 312 includes a distal end 348 and a proximal end 350. The cover sleeve 314 is also shown and includes a distal end 352 and a proximal end 354. These respective distal and proximal ends are, in the depicted embodiment, opposite each other. It will be appreciated that the inner shaft 308, middle shaft 310, outer shaft 312, and cover sleeve 314 are not drawn to scale in FIG. 19. Each of the inner shaft 308, middle shaft 310, outer shaft 312, and cover sleeve 314 has a suitable length for a thrombectomy procedure. For instance, the working length of the extractor device 300 is capable of reaching all the way to the inferior vena cava (IVC) bifurcation or iliac of the patient's anatomy in order to remove clot from the iliac all the way to the popliteal vein behind the patient's knee. In an example, the inner shaft 308 may have a length within a range of 140 cm to 145 cm. The middle shaft 310 may have a length within a range of 115 cm to 120 cm. The outer shaft 312 may have a length within a range of 100 cm to 105 cm. The cover sleeve 314 may have a length within a range of 70 cm to 90 cm. In an example, a wall thickness of each of the inner shaft 308, middle shaft 310, outer shaft 312, and cover sleeve 314 is within a range of 0.010 cm to 0.020 cm.

In various embodiments, each of the inner shaft 308, middle shaft 310, and outer shaft 312 is constructed of a material that includes a polymer, such as a polyimide. The polymer may be reinforced with a coil or braid to improve kink resistance and allow for greater directional control. The coil or braid may include a metal, such as steel, and more particularly such as stainless steel. Each of the inner shaft 308, middle shaft 310, and outer shaft 312 may include an inner liner configured to provide a low friction surface for another of the shafts or a guidewire to pass through. For example, in various embodiments, the inner liner may include a polymer, such as an elastomer, and more particularly such as a thermoplastic elastomer. For example, the inner liner may include polytetrafluoroethylene (PTFE), polyether block amide (PEBA) (e.g., PEBAX®), or PEBA with a slip additive (e.g., EverGlide®).

The cover sleeve 314 keeps the extractor catheter 332 collapsed (or in a constrained position) during insertion of the extractor device 300 into, through, and/or within a patient's vasculature so that, for example, the extractor device 300 can reach the iliac or IVC bifurcation without disturbing a clot that is in the iliofemoral popliteal section. The cover sleeve 314 is constructed with multiple layers similar to a typical guide catheter. For example, an outer jacket layer of the cover sleeve 314 may include a polymer, such as an elastomer, and more particularly such as a thermoplastic elastomer. For example, the polymer may include polyether block amide (PEBA) (e.g., PEBAX®), PEBA with a slip additive (e.g., EverGlide®), or Nylon 12. A reinforcing braid or coil layer may include a metal, such as steel, and more particularly such as stainless steel. An inner liner layer may include a polymer, such as an elastomer, and more particularly such as a thermoplastic elastomer. For example, the inner liner may include polytetrafluoroethylene (PTFE), polyether block amide (PEBA) (e.g., PEBAX®), or PEBA with a slip additive (e.g., EverGlide®). In some embodiments, a distal end of the cover sleeve 314 may include a hydrophilic coating.

In some embodiments, a proximal end of the cover sleeve 314 may include one or more depth markers. For example, the depth markers may be imprinted on the surface of the cover sleeve 314. In an example, the depth markers may indicate 5 cm intervals. The depth markers can be used by a physician to determine how far the physician has retracted the extractor catheter 332.

In the illustrated implementation, the extractor device 300 includes four flush ports 316A, 316B, 316C, and 316D. The flush port 316A enables flushing the space within the inner shaft 308. The flush port 316B includes a tube 356A (e.g., a flexible polymer tube) and allows for flushing the space between the inner shaft 308 and the middle shaft 310. The flush port 316C includes a tube 356B (e.g., a flexible polymer tube) and allows for flushing the space between the middle shaft 310 and the outer shaft 312. The flush port 316D includes a tube 356C (e.g., a flexible polymer tube) and allows for flushing the space between the outer shaft 312 and the cover sleeve 314. In some embodiments, each of the flush ports 316A, 316B, 316C, and 316D may be a Luer activated valve.

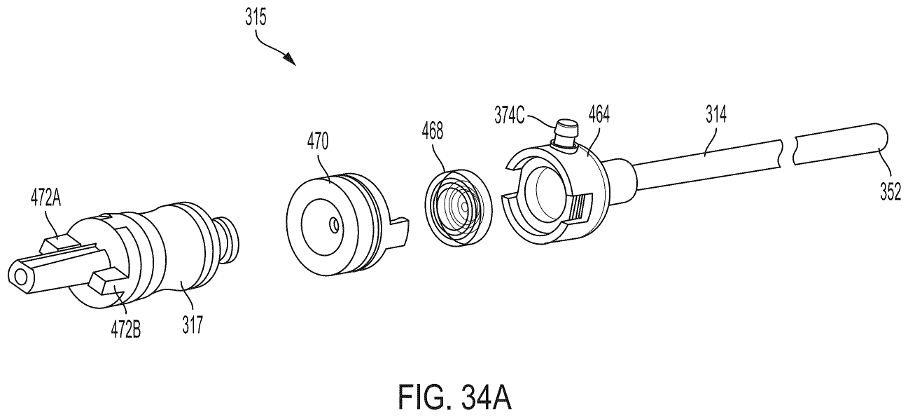

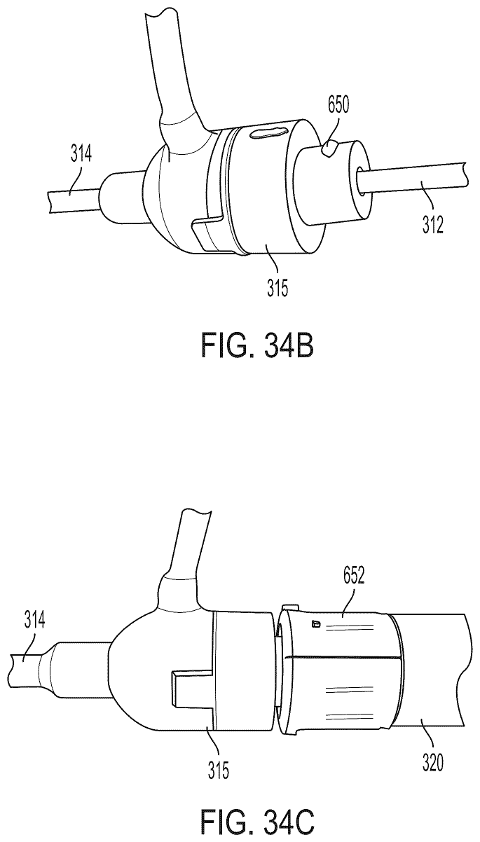

In at least some embodiments, the flush port 316D is included with a cover sleeve hub 315. The cover sleeve hub 315 can be manually advanced or retracted along the axis 343 to sheath or unsheathe the extractor catheter 332 with or from the cover sleeve 314. When the extractor catheter 332 is unsheathed from the cover sleeve 314, the cover sleeve hub 315 may be coupled to the sliding member 320. For example, with reference to FIGS. 34B and 34C, the cover sleeve hub 315 may include a protrusion 650 that snaps into an interior indentation of a clip 652 of the sliding member 320.

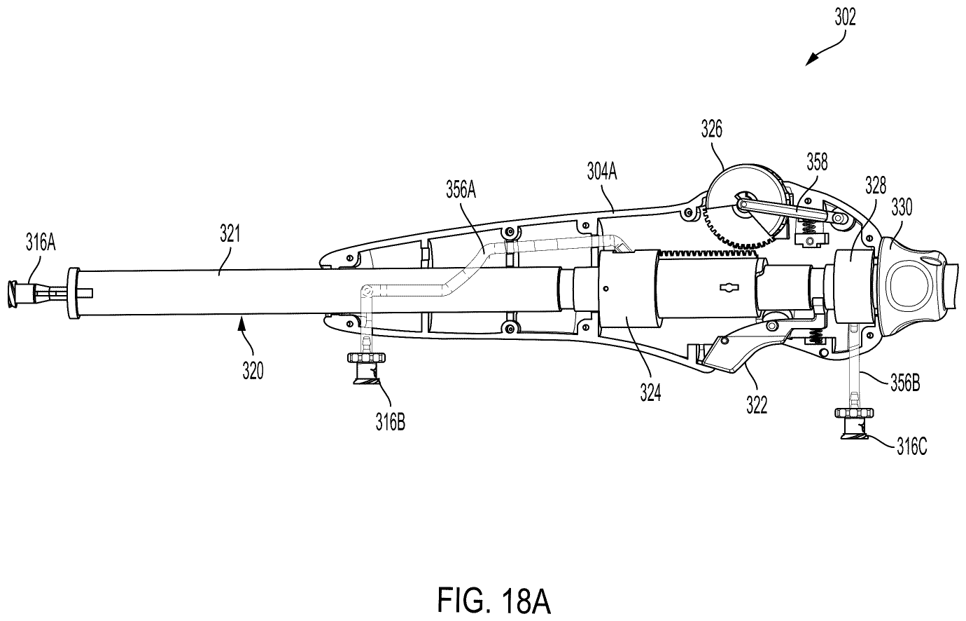

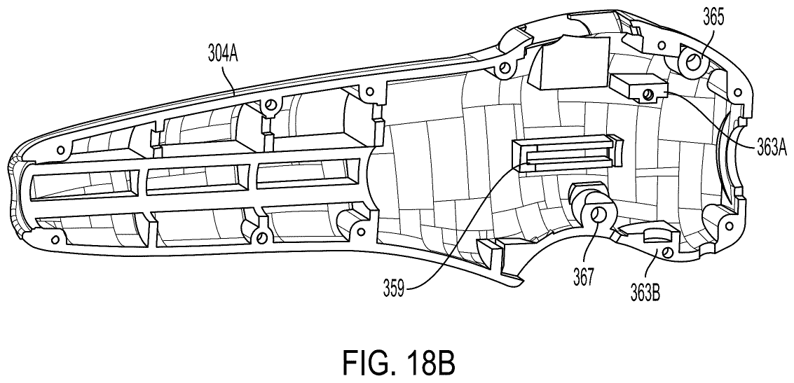

FIG. 18A is a side view of a portion of the handle 302 with half 304B of the housing 304 removed to reveal an example implementation of the interior of the housing 304. The half 304A of the housing 304 remains visible. The sliding member 320 is shown disposed through the housing 304. The trigger 322 is shown to have a portion extending outside the housing 304 and another portion contacting the sliding member 320. The middle shaft hub 324 is disposed around the sliding member 320 and is connected to the tube 356A of the flush port 316B. The outer shaft hub 328 is disposed around the sliding member 320 and is connected to the tube 356B of the flush port 316C. and pivot member 358 is pivotably coupled to the housing 304 and to the dial 326.

FIG. 18B is a perspective view of the half 304A of the housing 304 shown in FIG. 18A. The half 304A includes an elongated slot 359. The half 304A further includes a platform 363A and/or a platform 363B. Slots 365 and 367 are also included with the half 304A of the housing 304. The interior of the other half 304B of the housing 304, though not shown in the figures, is similar to (e.g., a mirror image of) the half 304A. In some embodiments, the housing 304 may be a single component rather than two separate halves 304A, 304B. For example, the housing 304 may be three-dimensionally printed around the components interior to the housing 304.

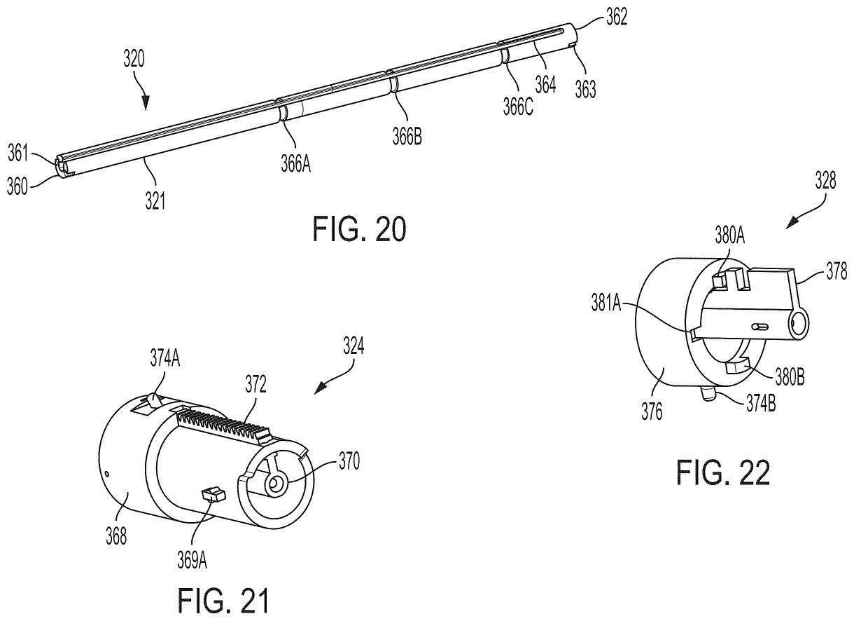

FIG. 20 illustrates the sliding member 320 in isolation. The sliding member 320 includes a proximal end 360 and a distal end 362, which, in the depicted embodiment, is opposite the proximal end 360. The distal end 362 includes at least one notch 373. A channel 361 extends through the sliding member 320 from the proximal end 360 to the distal end 362. A track 364 (e.g., an elongated opening) also extends along the sliding member 320 and is joined with the channel 361 such that an object of suitable shape and size can be within the track 364 and the channel 361 at the same time. The exterior of the sliding member 320 includes a plurality of notches 366A, 366B, 366C between the proximal and distal ends 360, 362.

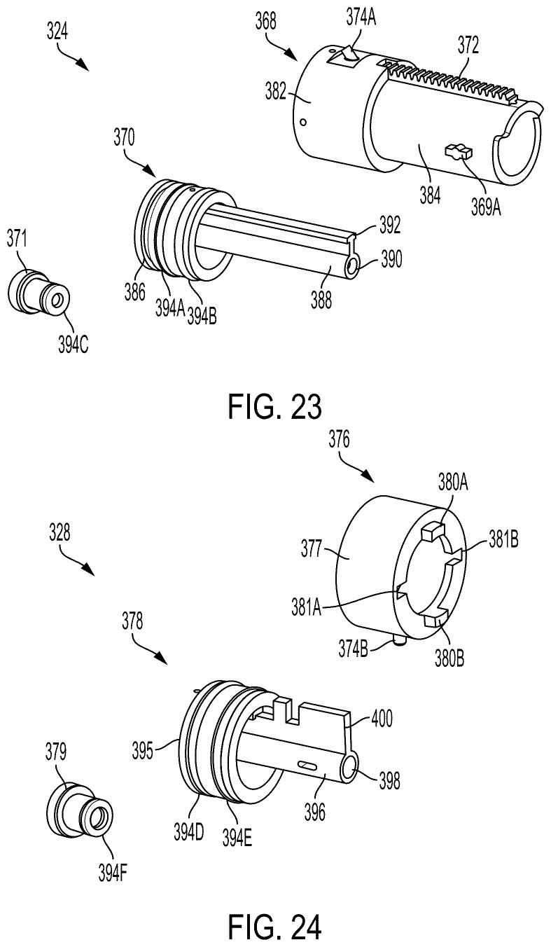

FIGS. 21 and 23 illustrate the middle shaft hub 324 in isolation. The middle shaft hub 324 includes an outer member 368, an inner member 370, and an end cap 371. The outer member 368 includes a first portion 382 and a second portion 384. The first portion 382 may include a stem 374A. The second portion 384 may include a protrusion 369A, and in some embodiments, a protrusion 369B (not illustrated) on the opposing side of the second portion 384 to the protrusion 369A. The protrusions 369A, 369B may be disposed within the respective slots 359 of the first half 304A and the second half 304B of the housing 304. In this way, the outer member 368 is rotationally fixed relative to the housing 304. The second portion 384 includes an engagement portion, which, in the depicted embodiment, comprises a plurality of teeth 372.

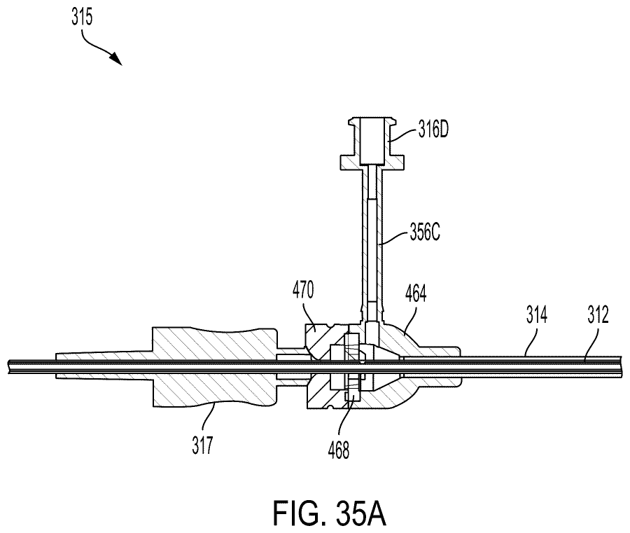

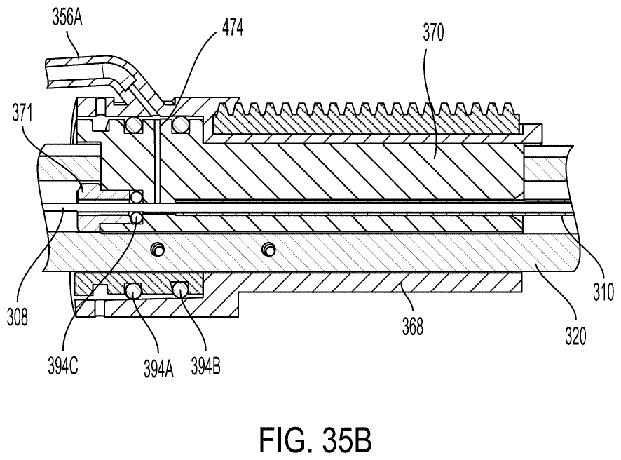

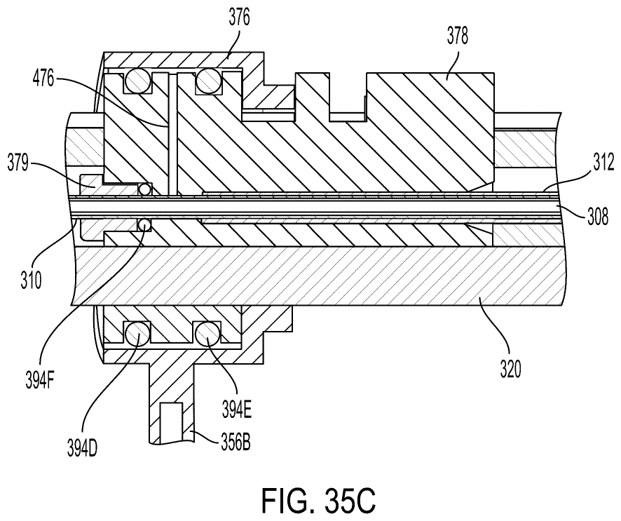

The inner member 370 includes a body 386 and an elongated member 388 extending from the body 386. The elongated member 388 forms a channel 390 and includes a coupling member 392. The body 386 includes at least one notch configured to accept at least one sealing member (e.g., an O-ring). For example, the inner member 370 is illustrated as having two sealing members 394A, 394B. The inner member 370 may be disposed within the outer member 368 such that the body 386 is disposed within the first portion 382. The end cap 371 includes a channel therethrough and is configured to be partially disposed with a sealing member 394C within the channel 390 on the end of the elongated member 388 within the body 386 (see FIG. 35B). The sealing members 394A, 394B, 394C contribute to flushing operations that will be described below.

FIGS. 22 and 24 illustrate the outer shaft hub 328 in isolation. The outer shaft hub 328 includes an outer member 376, an inner member 378, and an end cap 379. The outer member 376 includes a body 377 and a stem 374B. The body 377 may include protrusions 380A, 380B. The body 377 may additionally or alternatively include notches 381A, 381B. The inner member 378 includes a body 395 and an elongated member 396 extending from the body 395. The elongated member 396 forms a channel 398 and includes a coupling member 400. The body 395 includes at least one notch configured to accept at least one sealing member (e.g., an O-ring). For example, the inner member 378 is illustrated having two sealing members 394D, 394E. The inner member 378 may be disposed within the outer member 376 such that the body 395 is disposed within the body 377 of the outer member 376. The end cap 379 includes a channel therethrough and is configured to be partially disposed, together with a sealing member 394F, within the channel 398 on the end of the elongated member 396 within the body 395 (see FIG. 35C). The sealing members 394D, 394E, 394F contribute to flushing operations that will be described below.

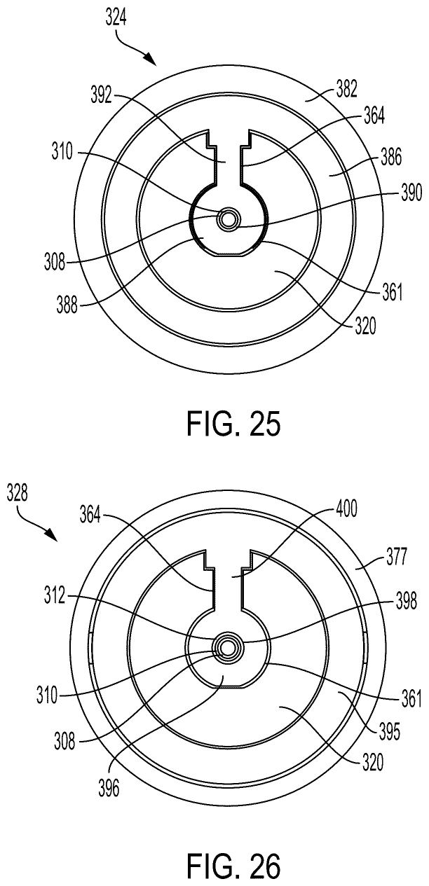

FIG. 25 is a cross-sectional slice of the middle shaft hub 324 at the first portion 382 of the outer member 368. As shown, the body 386 of the inner member 370 is disposed within the first portion 382 of the outer member 368. The sliding member 320 is shown disposed within the body 386. Additionally, the elongated member 388 of the inner member 370 is disposed within the sliding member 320. Specifically, the elongated member 388 is disposed within the channel 361 of the sliding member 320, with the coupling member 392 disposed within the track 364 of the sliding member 320. The coupling member 392 couples rotational movement of the sliding member 320 and the inner member 370 of the middle shaft hub 324. Based on the position of the middle shaft hub 324 in the extractor device 300, the inner shaft 308 and the middle shaft 310 extend through the cross-sectioned portion of the channel 390 of the middle shaft hub 324, but the outer shaft 312 does not.

FIG. 26 is a cross-sectional slice of the outer shaft hub 328 at a portion of the outer member 376. As shown, the body 395 of the inner member 378 is disposed within the outer member 376. The sliding member 320 is shown disposed within the body 395. Additionally, the elongated member 396 of the inner member 378 is disposed within the sliding member 320. Specifically, the elongated member 396 is disposed within the channel 361 of the sliding member 320, with the coupling member 400 disposed within the track 364 of the sliding member 320. The coupling member 392 couples rotational movement of the sliding member 320 and the outer shaft hub 328. Based on the position of the outer shaft hub 328 in the extractor device 300, the inner shaft 308, the middle shaft 310, and outer shaft 312 all extend through the cross-sectioned portion of the channel 398 of the outer shaft hub 328.

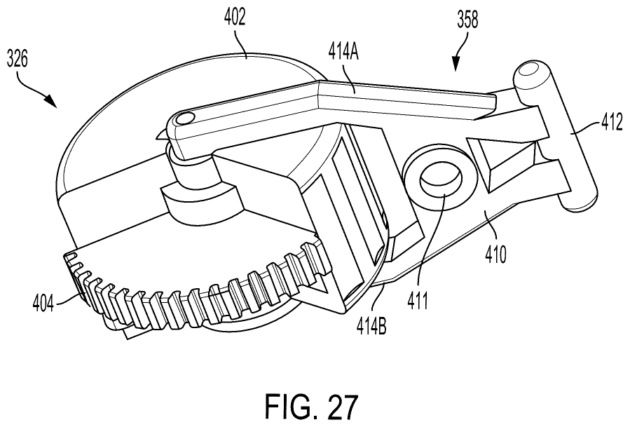

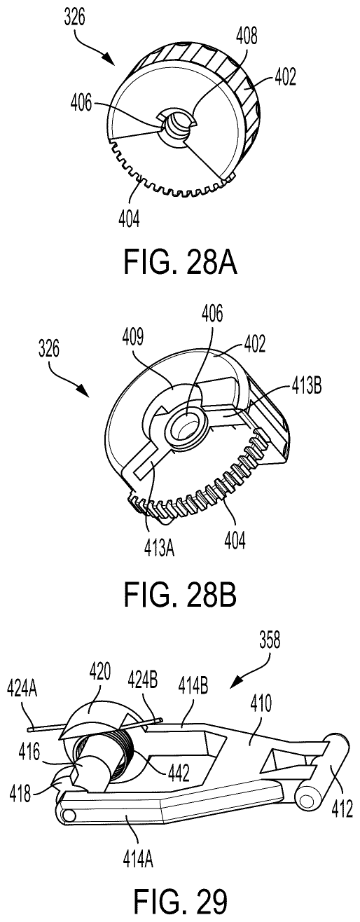

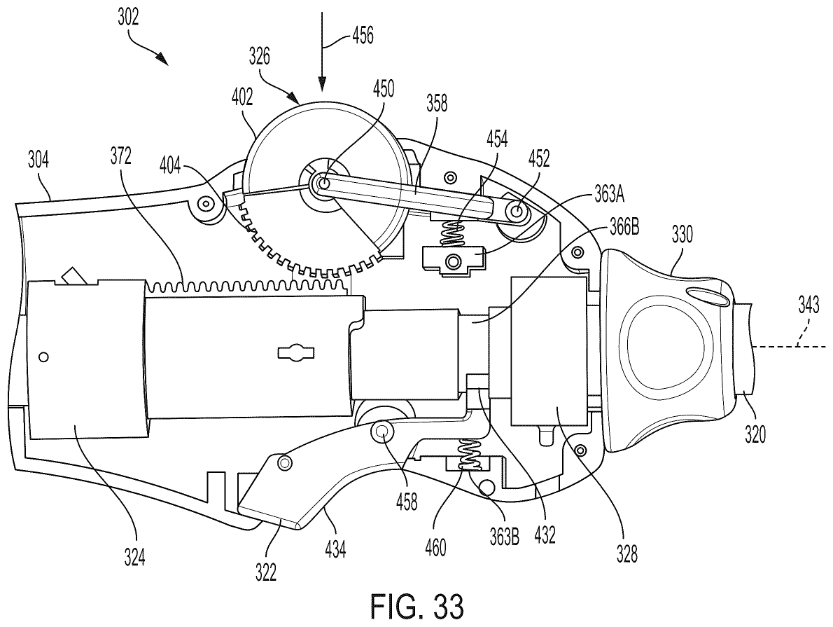

FIGS. 27 to 29 illustrate the dial 326 and the pivot member 358. The dial 326 includes a control portion 402 and an engagement portion, which, in the depicted embodiment, comprises a plurality of teeth 404. The control portion 402 may be a separate component from the plurality of teeth 404 or may be integral with the plurality of teeth 404. A channel 406 extends through the dial 326. A first side (see FIG. 28A) of the control portion 402 includes a notch 408. The second, opposing side (see FIG. 28B) includes an opening 409 and slots 413A, 413B. The pivot member 358 includes a body 410 having a surface 411. The surface 411 is contacted by a resilient member 454 (FIG. 33) when the extractor device 300 is fully constructed, which will be described below. The body 410 includes arms 414A, 414B connected to each side of a pivot bar 416 of the body 410. The pivot bar 416 extends through the channel 406 of the dial 326 when the pivot member 358 is coupled to the dial 326. The pivot bar 416 includes a protrusion 418 that matches the shape of, and is disposed within, the notch 408 of the control portion 402 of the dial 326. The arm 414B includes a protrusion 420 that extends into the opening 409 of the control portion 402. The body 410 may also include a pivot bar 412. The ends of the pivot bar 412 may be disposed within the respective slots 365 of the first half 304A and the second half 304B of the housing 304. In this way, the pivot member 358 may pivot about an axis extending through the pivot bar 412 relative to the housing 304.

A torsion spring 422 may be used to bias the dial 326 toward a starting position. For example, the torsion spring 422 may be disposed around the pivot bar 416 and includes a first end portion 424A and a second end portion 424B. When the pivot member 358 is coupled to the dial 326, the first end portion 424A is disposed within the slot 410B of the dial 326 and the second end portion 424B is disposed within the slot 410A. In this way, rotating the dial 326 via the control portion 402 requires overcoming the force applied by the torsion spring 422. When the control portion 402 is released, the torsion spring 422 returns the dial 326 back to the original starting position.

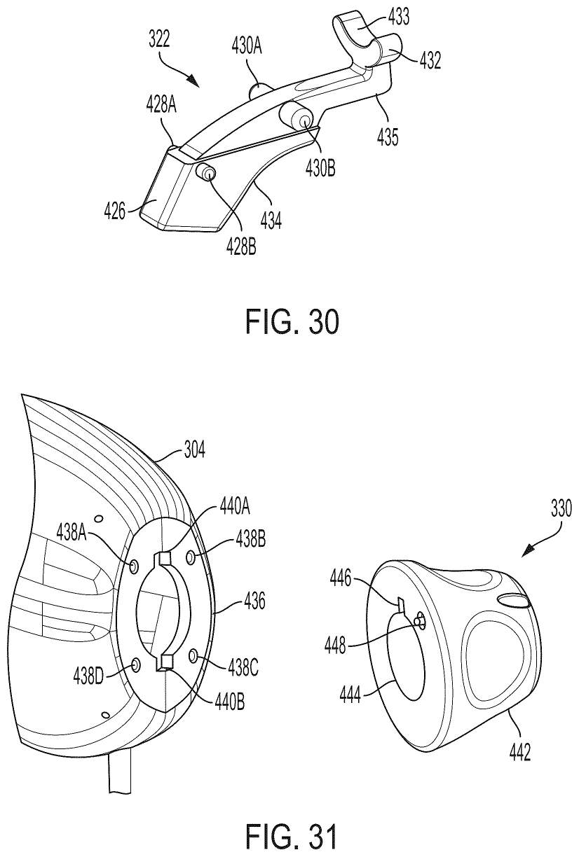

FIG. 30 is a perspective view of the trigger 322. The trigger 322 includes a body 426. Protrusions 428A, 428B, as well as protrusions 430A, 430B, extend from the body 426. The protrusions 430A, 430B may be disposed within the respective slots 367 of the first half 304A and the second half 304B of the housing 304. The body 426 may further include a jaw 432 extending from a distal end 435 of the body 426. The jaw 432 includes a curved surface 433. The curved surface 433 may have a similar (e.g., the same) radius of curvature as the exterior surface of the sliding member 320. The body 426 further includes a contact portion 434. A physician contacts the contact portion 434 when activating or releasing the trigger 322.

FIG. 31 is a perspective view of the knob 330 shown in relation to a distal end 436 of the housing 304. The knob 330 includes a body 442 defining a channel 444 through which the sliding member 320 may extend. The body 442 further defines a slot 446. The slot 446 is sized to accept the coupling member 378 of the outer shaft hub 328. A protrusion 448 may extend from the body 442. The protrusion 448 may be integral with the body 442 or may be coupled to the body 442. The distal end 436 of the housing 304 defines a plurality of indentations 438A, 438B, 438C, 438D that are each configured to receive the protrusion 448 of the knob 330. When the protrusion 448 is received in one of the indentations 438A, 438B, 438C, 438D, the knob 330 is discouraged or prevented from rotating about the axis 343. The distal end 436 of the housing 304 may further define slots 440A, 440B. The slots 440A, 440B are sized to accept the protrusions 380A, 380B of the outer shaft hub 328.

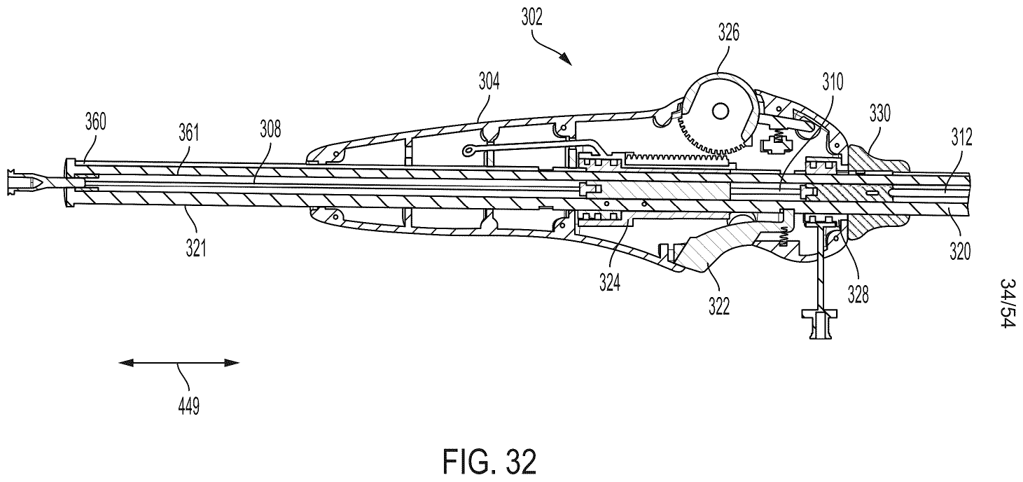

Referring now to FIG. 32, the cross-section shows the sliding member 320 extending through the middle shaft hub 324, the outer shaft hub 328, and the knob 330. With this configuration, the sliding member 320 can translate relative to the middle shaft hub 324, the outer shaft hub 328, and the knob 330 in either direction of the double-sided arrow 449 along the axis 343. Additionally, the proximal end 350 of the outer shaft 312 is shown to be coupled to the outer shaft hub 328. For instance, the outer shaft 312 is visible distal to the outer shaft hub 328, but not proximal to the outer shaft hub 328. The proximal end 346 of the middle shaft 310 is also shown to be coupled to the middle shaft hub 324. For instance, the middle shaft 310 is visible distal to the middle shaft hub 324, but not proximal to the middle shaft hub 324. The proximal end 342 of the inner shaft 308 is shown to be coupled to the proximal end 360 of the sliding member 320.

Operations of the dial 326, the trigger 322, and the knob 330 will now be described in relation to FIG. 33. The pivot member 358 is coupled to the dial 326. The pivot member 358 may pivot relative to the housing 304 about pivot axis 452, and also relative to the dial 326 about pivot axis 450. A resilient member 454 is disposed between the platform 363A of the housing 304 and the surface 311 of the pivot member 358. In this way, the dial 326, and particularly the plurality of teeth 404 of the dial 326, are biased away from the plurality of teeth 372 of the middle shaft hub 324 such that the dial 326 is in a disengaged position at rest. A physician may press on the control portion 402 of the dial 326 in the direction of the arrow 456, with sufficient force to overcome the biasing force of the resilient member 454, to engage at least some of the plurality of teeth 404 with at least some of the plurality of teeth 372 and thus engage the dial 326 with the middle shaft hub 324. With the dial 326 in the engaged position, in which at least some of each of the plurality of teeth 404 and 372 are engaged (e.g., a male tooth of one plurality is engaged with a female tooth of the other plurality), the physician may rotate the dial 326 to cause the middle shaft hub 324 to translate along the axis 343, which causes the middle shaft 310 to translate along the axis 343. When the physician releases the dial 326, the biasing force of the resilient member 454 returns the dial 326 to the disengaged position such that the plurality of teeth 404 and 372 are disengaged.

The trigger 322 is shown in an engaged position in which the curved surface 433 of the jaw 432 of the trigger 322 contacts the notch 366B of the sliding member 320. With the trigger 322 in the engaged position, the sliding member 320 is prevented from translating along the axis 343. A resilient member 460 extends between a proximal end of the trigger 322 and the platform 363B of the housing 304. In this way, the trigger 322 is biased toward the engaged position. A physician may squeeze the contact portion 434 of the trigger 322 with sufficient force to overcome the biasing force of the resilient member 460 such that the trigger 322 pivots about the pivot axis 458 into a disengaged position. The jaw 432 is spaced away from the notch 366B of the sliding member 320 with the trigger in the disengaged position such that the sliding member 320 has clearance to translate along the axis 343. When the physician releases the trigger 322, the resilient member 460 pivots the trigger 322 back to the engaged position. If the jaw 432 is not aligned with a notch 366A, 366B, 366C after the trigger 322 is released, the sliding member 320 may be translated until the jaw 432 snaps into a notch 366A, 366B, 366C.

The knob 330 is shown in an engaged position with the protrusion 448 of the knob 330 disposed in one of the indentations 438A, 438B, 438C, 438D of the housing 304. The physician may transition the knob 330 to a disengaged position by gripping the body 442 of the knob 330 and translating the knob 330 along the axis 343 away from the housing 304 until the protrusion 448 has clearance from the indentation 438A, 438B, 438C, or 438D. With the knob 330 in the disengaged position, the physician may rotate the knob 330 about the axis 343 until the protrusion 448 is aligned with a selected indentation 438A, 438B, 438C, or 438D. The physician may then translate the knob 330 towards the housing 304 to return the knob 330 back to the engaged position with the protrusion 448 disposed in the selected indentation 438A, 438B, 438C, or 438D. Because the coupling member 378 of the outer shaft hub 328 is disposed within the slot 446 of the knob 330, rotating the knob 330 rotates the outer shaft hub 328, which rotates the sliding member 320 because the coupling member 378 is further disposed within the track 364 of the sliding member 320, which rotates the middle shaft hub 324 because the coupling member 392 of the middle shaft hub 324 is also disposed within the track 364 of the sliding member 320. In this way, rotating the knob 330 rotates the inner shaft 308, the middle shaft 310, and the outer shaft 312.

Referring now to FIG. 34A, an exploded view of the cover sleeve hub 315 is shown. The cover sleeve hub 315 includes a base member 464 from which the cover sleeve 314 extends. A stem 374C for connecting the base member 464 to the tubing 356C additionally extends from the base member 464. A sealing member 468 is disposed between the base member 464 and an end cap 470 that may be coupled to the base member 464. A connector 317 may be coupled to the end cap 470. The connector 317 includes protrusions 472A, 472B that may engage with the at least one notch 373 of the sliding member 320 to couple the connector 317 to the sliding member 320.

The flushing capabilities of the extractor device 300 will now be described. Flushing is a standard procedure with all medical devices that enter a patient's body in order to remove any air that is trapped within the device and, if introduced into the patient, could lead to complications such as an air embolism. Medical devices are typically flushed with saline solution, though another suitable flushing fluid may be used. As described above, the extractor device 300 includes four flush ports 316A, 316B, 316C, and 316D. The flush port 316A enables flushing the space within the inner shaft 308, such as between a guidewire and the inner surface of the inner shaft 308. The flush port 316B allows for flushing the space between the inner shaft 308 and the middle shaft 310, the flush port 316C allows for flushing the space between the middle shaft 310 and the outer shaft 312, and the flush port 316D allows for flushing the space between the outer shaft 312 and the cover sleeve 314.

With reference to FIG. 35A, the cover sleeve hub 315 is fluidly connected to the flush port 316D via the tube 356C. The sealing member 468 guides the saline solution from the tube 356C into the space between the outer shaft 312 and the cover sleeve 314. For instance, the sealing member 468 prevents the flushing (e.g., saline) solution from flowing past the sealing member 468 toward the sliding member 320. In this way, any air trapped in the space between the outer shaft 312 and the cover sleeve 314 is flushed out.

With reference to FIG. 35B, the middle shaft hub 324 is fluidly connected to the flush port 316B via the tube 356A. The O-rings 394A, 394B guide the saline solution from the tube 356A into a channel 474 of the inner member 370. For instance, the O-rings 394A, 394B maintain the saline solution between the O-rings 394A, 394B. Once the saline solution exits the channel 474, the O-ring 394C guides the saline solution into the space between the inner shaft 308 and the middle shaft 310. For instance, the O-ring 394C prevents the saline solution from flowing past the O-ring 394C toward the proximal end 342 of the inner shaft 308. The O-rings 394A, 394B, and 394C minimize the saline solution leaking into the housing 304. In this way, any air trapped in the space between the inner shaft 308 and the middle shaft 310 is flushed out.

With reference to FIG. 35C, the outer shaft hub 328 is fluidly connected to the flush port 316C via the tube 356B. The O-rings 394D, 394E guide the saline solution from the tube 356B into a channel 476 of the inner member 378. For instance, the O-rings 394D, 394E maintain the saline solution between the O-rings 394D, 394E. Once the saline solution exits the channel 476, the O-ring 394F guides the saline solution into the space between the middle shaft 310 and the outer shaft 312. For instance, the O-ring 394F prevents the saline solution from flowing past the O-ring 394F toward the proximal end 346 of the middle shaft 310. The O-rings 394D, 394E, and 394F minimize the saline solution leaking into the housing 304. In this way, any air trapped in the space between the middle shaft 310 and the outer shaft 312 is flushed out.

An example flushing method that may be performed includes the following. Before flushing begins, the extractor catheter 332 is in a collapsed state. For example, when the extractor catheter 332 is in the collapsed state, each of the inner shaft 308 and the middle shaft 310 are advanced far enough away from the handle 302 that the mesh 334 and the coring element 336 are extended so as to be close enough to the inner shaft 308 and the middle shaft 310 that the cover sleeve 314 can be advanced over the mesh 334 and the coring element 336.

Flushing begins by introducing saline solution into the flush port 316B to flush the space between the inner shaft 308 and the middle shaft 310. Then, saline solution is introduced into the flush port 316C to flush the space between the middle shaft 310 and the outer shaft 312. Then, saline solution is introduced into the flush port 316D to flush the space between the outer shaft 312 and a cover sleeve 314. As soon as the space between the outer shaft 312 and a cover sleeve 314 is flush, the cover sleeve 314 is advanced forward to sheath the extractor catheter 332. Then, saline solution is introduced into the flush port 316A to flush the space between a guidewire and the inner shaft 308.

### Extractor Catheter Embodiments

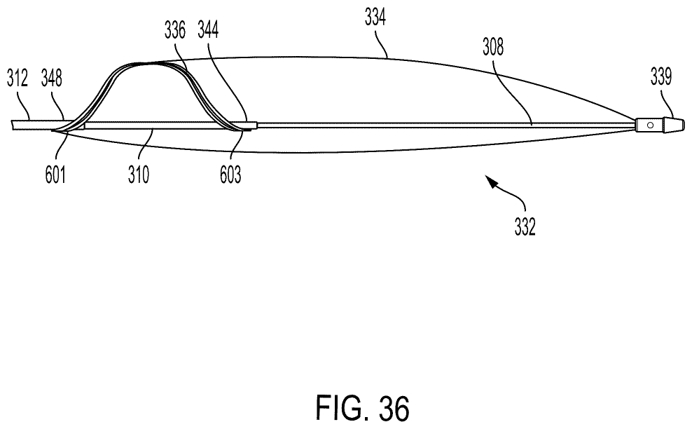

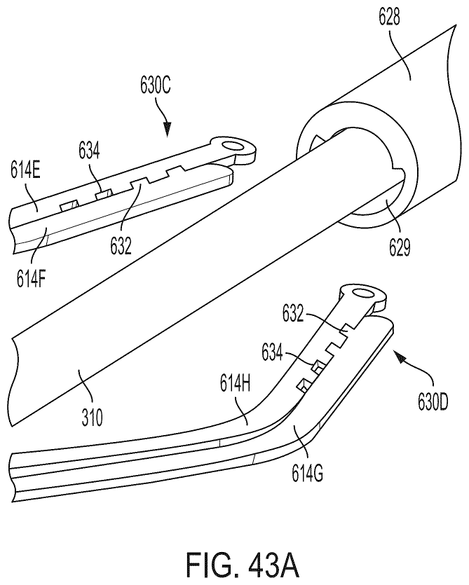

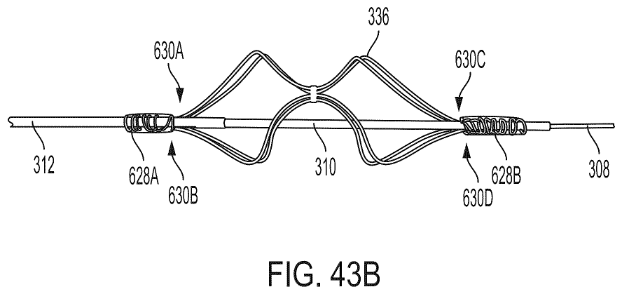

FIG. 36 depicts a distal end of the extractor device 300 showing a magnified view of the extractor catheter 332. The extractor catheter 332 includes the mesh 334, the coring element 336, and a distal tip 339. A proximal end 601 of the coring element 336 is coupled to the distal end 348 of the outer shaft 312. The outer shaft 312 serves as an anchor for the coring element 336 and as the outer structural support for the shaft assembly 306. A distal end 603 of the coring element 336 is coupled to the distal end 344 of the middle shaft 310. In this way, movement of the middle shaft 310 is coupled to the opening and closing of the coring element 336 and thereby the opening and closing of the proximal end of the mesh 334. This end of the mesh 334 is where clot material enters the mesh and may therefore be characterized as the mouth of the mesh 334. Examples of how the coring element 336 can be coupled to the outer shaft 312 and the middle shaft 310 are described below in connection with FIGS. 43A and 43B.

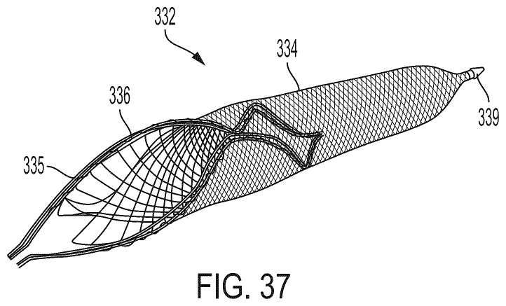

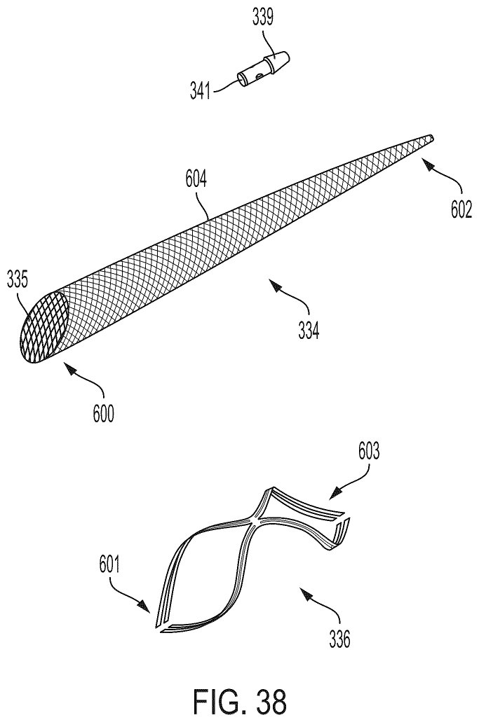

FIGS. 37 and 38 illustrate the extractor catheter 332 in isolation. The mesh 334 includes a proximal end 600 and a distal end 602. The proximal end 600 forms a mouth 335 of the mesh 334 that is open whereas the distal end 602 is closed, such as by the distal tip 339. The distal tip 339 includes a channel 341 extending therethrough. Referring again to FIG. 36, the distal tip 339 is coupled to the inner shaft 308 and to the distal end 602 of the mesh 334. In this way, movement of the inner shaft 308 is coupled to movement of the distal end 602 of the mesh 334. The proximal end 600 of the mesh 334 is coupled to a proximal portion of the coring element 336.

The mesh 334 may include a plurality of wires braided together. The wires may include nitinol, another suitable shape-memory material, or another suitable material. In the example embodiments described herein, the mesh 334 is treated as including a shape-memory material. In some embodiments, at least some of the wires have a diameter within a range of 0.010 cm to 0.016 cm. In one example, the mesh 334 may be formed using a 48-carrier braider with a full load (i.e., 48 wires) configuration. The mesh 334 may be made of a single or multiple picks per inch (PPI) segments. In various embodiments, the mesh 334 may have a length from the proximal end 600 and the distal end 602 within a range of 5 cm to 19 cm, though other lengths and ranges of lengths are possible. In at least some embodiments, at least one of the wires of the mesh 334 includes a radiopaque material, such as platinum, which facilitates the position of the mesh 334 being visible during fluoroscopy. In some embodiments, mesh 334 can be made with an elastomer polyurethane cover on the distal end 602 (or an even greater distal portion) of the mesh 334 to prevent embolic loss.

In some embodiments, the proximal end 600 and distal end 602 of the mesh 334 each have a smaller pore size than a middle portion 604 of the mesh 334. For example, the pore size of the proximal end 600 and distal end 602 may be greater than 0 and less than or equal to 2 millimeters (mm), whereas the pore size of the middle portion 604 may be greater than 2 mm and less than or equal to 5 mm. The larger pore size in the middle portion 604 of the mesh 334 may facilitate excess clots being broken up and squeezed through the pores to exit the mesh 334 if the mesh 334 becomes too full. The average pore size of the proximal end 600 may be similar to the distal end 602 or may be different.

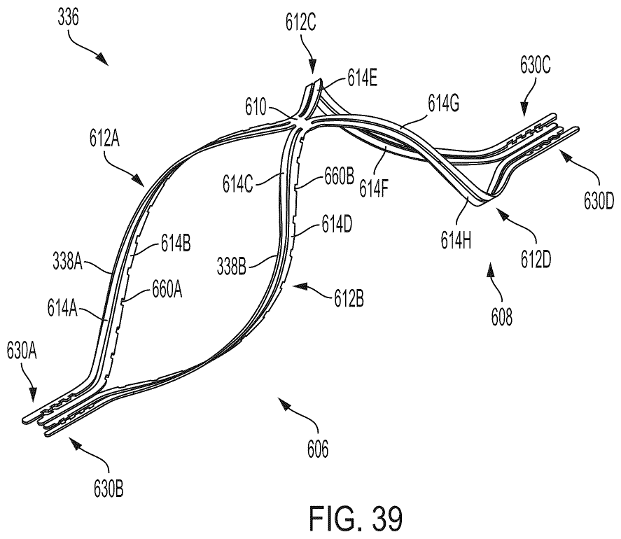

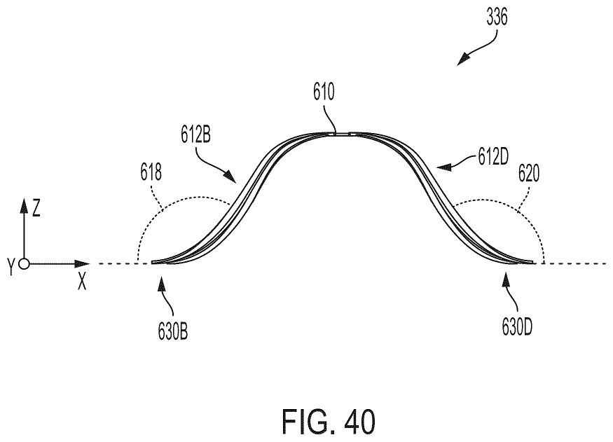

Referring to FIGS. 39 and 40, the coring element 336 includes a first portion 606, which is an example of a proximal portion of the coring element 336, and a second portion 608, which is an example of a distal portion of the coring element 336. The first portion 606 may be joined to (e.g., integrally formed with) the second portion 608 by a bridge 610, which is an example of a central segment, region, or portion to which the arms of the first and second portions 606, 608 are connected and from which those arms extend. In some embodiments, the shape of the first portion 606 may be a mirror image of the shape of the second portion 608 relative to the bridge 610. The first portion 606 includes a first set of arms 612A and a second set of arms 612B. The first set of arms 612A includes an arm 614A and an arm 614B. The second set of arms 612B includes an arm 614C and an arm 614D. The embodiment of the coring element 336 shown in FIG. 39 is drawn to scale, meaning the sizes of the depicted features thereof are accurate relative to each other for at least the embodiment depicted in FIG. 39; but as a person of ordinary skill in the art will understand, the present disclosure is not limited to the depicted embodiment of the coring element, and other embodiments that do not possess the depicted sizes also fall within the scope of the relevant claims.

A first pair of arms 614A, 614C form a blade or cutting edge that assists in breaking down blood clots and separating adhered blood clots from a vessel wall. For instance, the arm 614A includes a blade (or cutting edge) portion 338A and the arm 614C includes a blade (or cutting edge) portion 338B. A second pair of arms 614B, 614D are disposed closer to the second portion 608 than are the first pair of arms 614A, 614C. The second pair of arms 614B, 614D include a plurality of notches. For instance, the arm 614B includes a plurality of notches 660A and the arm 614D includes a plurality of notches 660B. The notches 660A, 660B may be used for securing (e.g., directly coupling) the second pair of arms 614B, 614D to the mesh 334, as described further below. In some embodiments, the notches 660A, 660B may be omitted. Distal ends 630A, 630B of the first and second sets of arms 612A, 612B may be coupled to the outer shaft 312 of the handle 302.

The second portion 608 of the coring element 336 includes a third set of arms 612C and a fourth set of arms 612D. The third set of arms 612C includes an arm 614E and an arm 614F. The fourth set of arms 612D includes an arm 614G and an arm 614H. The arms in the third pair of arms 614E, 614G are disposed farther from the first portion 606 than are the arms in the fourth pair of arms 614F, 614H. Distal ends 630C, 630D of the third and fourth sets of arms 612C, 612D may be coupled to the middle shaft 310 of the handle 302.

Each of the arms 614A-H is formed in an at least partially-twisted ribbon-like shape. For instance, near the bridge 610, a first side of the arm 614A faces out-of-the-page whereas that first side is facing into-the-page at the end of the arm 614A away from the bridge 610. Additionally, near the bridge 610, the arm 614A is the inner arm relative to the arm 614B whereas at the end of the arm 614A away from the bridge 610 the arm 614A is the outer arm relative to the arm 614B.

In various embodiments, the coring element 336 may include nitinol or another material suitable for the utility described herein of the coring element 336. In at least some embodiments, the coring element 336 may be cut (e.g., laser cut) out of a tube (e.g., a hypo tube) or a sheet. Each of the arms 614A to 614H may have a cross-sectional thickness within a range of 0.012 to 0.018 inches (0.030 to 0.046 cm) and a width within a range of 0.040 to 0.080 inches (0.101 to 0.204 cm). The thickness and width of each of the arms 614A to 614H may all be the same, or some of the arms 614A to 614H may have different thicknesses and/or widths than other arms 614A to 614H. The blade portions 338A, 338B of the arms 614A, 614C may have a thickness within a range of 0.008 to 0.012 inches (0.020 to 0.031 cm). In other words, the blade (or cutting edge) portions of the arms 614A, 614C may have a thickness that is less than a thickness of another portion (e.g., the rest of) the arms 614A, 614C. In at least some embodiments, the coring element 336 may be heat treated. In at least some embodiments, each arm has a width that is greater than its thickness, including, in the depicted embodiment of the coring element 336, over the entire length of the arm except for a very short portion nearest the terminus of each arm (e.g., 90%-99% or more of the length of each arm).

The coring element 336 is configured such that each arm of the first set of arms 612A and the second set of arms 612B is angled. For instance, with reference to FIG. 40, a first plane including the x- and z-axes divides (e.g., bisects) the coring element 336 into a first side including the first set of arms 612A and the third set of arms 612C, and a second side including the second set of arms 612B and the fourth set of arms 612D. A second plane including the y- and z-axes (i.e., into and out of the page) divides (e.g., bisects) the coring element 336 into the first portion 606 and the second portion 608. A third plane including the x- and y-axes (i.e., a plane perpendicular to the first and second planes) forms an angle 618 with the first and second sets of arms 612A, 612B.

While the angle 618 is shown to be measured relative to tips of the blade portions in FIG. 40, the angle 618 may alternatively be measured from a portion of the arms 614A, 614C or from a portion of the arms 614B, 614D. The angle 618 is greater than 900 and less than 180°. For example, the angle 618 may be within one of the following ranges: 120 to 165°, 125 to 165°, 125 to 160°, 130 to 160°, 135 to 160°, 130 to 155°, 130 to 150°, 135 to 155°, or 135 to 150°. Additionally, with the proximal end 600 of the mesh 334 secured to the arms 614B, 614D of the coring element 336 in the constructed extractor catheter 332, the proximal end 600 of the mesh 334 also forms the angle 618 with the third plane including the x- and y-axes. Similar to the first and second sets of arms 612A, 612B, the third and fourth sets of arms 612C, 612D may form an angle 620 with the third plane including the x- and y-axes. The description of the angle 618 applies similarly to the angle 620. In some embodiments, the angle 618 may be equal to the angle 620, though the angles 618, 620 are not limited to these embodiments and may be unequal, including for different arms.

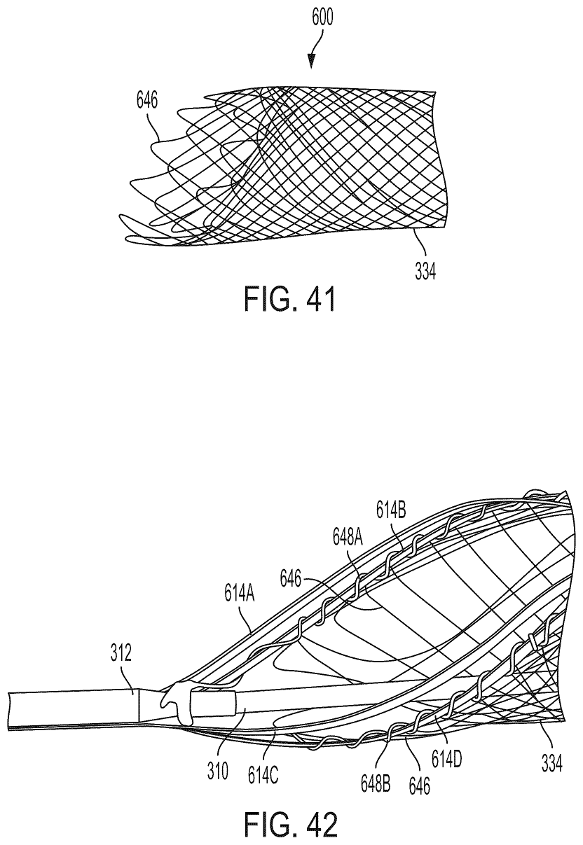

The connection of the mesh 334 to the coring element 336 will now be described. In some embodiments, the proximal end 600 of the mesh 334 may be secured (though not immovably) to the arms 614B, 614D via at least one wound wire, suture, or other suitable securing element. For example, FIG. 41 shows the proximal end 600 of the mesh 334 including a plurality of end loops 646. The plurality of end loops 646 may be secured to the arms 614B, 614D by at least one wire (e.g., a nitinol wire) wound around the arms 614B, 614D (but not the arms 614A, 614C) and through at least one or multiple of (e.g., each of) the plurality of end loops 646. For example, FIG. 42 shows a wire 648A wound around the arm 614B and through a first portion of the plurality of end loops 646, and a wire 648B wound around the arm 614D and through a second portion of the plurality of end loops 646. In some embodiments, the wires 648A, 648B may be a single wire that is wound through both the first and second portions of the plurality of end loops 646. Securing the proximal end 600 of the mesh 334 to the arms 614B, 614D in this way discourages the mesh 334 from shifting proximally during use.

In embodiments in which the arms 614B, 614D include the plurality of notches 660A, 660B, the wires 648A, 648B may be wound around the arms 614B, 614D such that at least a portion of the wires 648A, 648B is disposed in at least one of the plurality of notches 660A, 660B and, more preferably, such that at least a portion of the wires 648A, 648B is disposed in at least each of the plurality of notches 660A, 660B. Used in this way, The plurality of notches 660A, 660B discourage the wires 648A, 648B from sliding along the arms 614B, 614D. Used in this way, the plurality of notches 660A, 660B also discourage the mesh 334 from shifting proximally during use. The at least one wound wire may further be attached to the outer shaft 312 in any suitable fashion, including through the use of an adhesive. In other embodiments, one or more strands that are not wires (e.g., that are formed from suture material) may be used instead of the at least one wire to secure the proximal end of the mesh to a proximal portion (e.g., arms 614B, 614D) of the coring element.

Securement of the coring element 336 to the middle shaft 310 and the outer shaft 312 will now be described. In some embodiments, the first portion 606 of the coring element 336 may be coupled to the outer shaft 312, and the second portion 608 to the middle shaft 310, via respective caps. For example, FIG. 43A shows an embodiment in which a portion of the distal end of the middle shaft 310 is to be disposed within a cap 628. The cap 628 may include a metal or another suitable material. The cap 628 includes a gap 629 sized to accept the distal ends 630C, 630D of the third and fourth sets of arms. For example, the distal ends 630C, 630D of the third and fourth sets of arms may be positioned adjacent the a portion of the distal end of the middle shaft 310 and the cap 628 may be disposed over (e.g., forced over) those distal ends. Alternatively, the cap 682 may be positioned adjacent the distal end of the middle shaft 310 and the distal ends 630C, 630D may be forced into the gap 629. An adhesive joins together the middle shaft 310, the third and fourth sets of arms 612C, 612D, and the cap 628.

In some embodiments, the third and fourth sets of arms 612C, 612D (e.g., the distal ends 630C, 630D) each include engagement structures, such as respective opposing teeth. For example, the arm 614G is shown having male teeth 632 and the arm 614H is shown having female teeth 634. Interlocking of the male teeth 632 with the female teeth 634 fixes the positions of the arms 614G, 614H relative to one another. The arms 614F and 614E are similarly shown having male teeth 632 and female teeth 634, respectively. In some embodiments, one male tooth engaging one female tooth will suffice for the depicted purpose.

In some embodiments, instead of or in addition to the male and female teeth 632, 634, the distal end 630C of the third set of arms 612C and the distal end 630D of the fourth set of arms 612D are intertwined to coil together around the distal end 344 of the middle shaft 310. In some aspects of these embodiments, the arms 614G, 614F of the third set of arms 612C are intertwined with one another and/or the arms 614G, 614H of the fourth set of arms 612D are intertwined with one another. The cap 628 may similarly be positioned over the coiled sets of arms 612C, 612D, and adhesive similarly applied.

In some embodiments, the cap 628 may be a polymer extrusion or a heat-shrink polymer sleeve positioned over the distal ends 630C, 630D, and over a portion of the distal end of the middle shaft 310. In other embodiments, the cap 628 may be suture that is wrapped over the distal ends 630C, 630D, and over a portion of the distal end of the middle shaft 310 to secure those distal ends together. Adhesive is added to further strengthen the bond in such other embodiments.

The distal ends 630A, 630B of the first and second sets of arms 612A, 612B of the first portion 606 of the coring element 336 may be coupled to the outer shaft 312 in a similar manner as described above for coupling the distal ends 630C, 630D of the third set of arms 612C and the fourth set of arms 612D to the middle shaft 310. For example, FIG. 43B shows the distal ends 630A, 630B coupled to the outer shaft 312 by a cap 628A, and the distal ends 630C, 630D coupled to the middle shaft 310 by a cap 628B. In this example, the caps 628A and 628B are each a heat-shrink polymer sleeve.

Interaction of the extractor catheter 332 and the handle 302 will now be described. A patient's vasculature decreases in diameter from the IVC bifurcation (˜22-25 mm) to the common iliac vessel (˜13-16 mm) to the popliteal vessel (˜6 mm). In some instances, the handle 302 may be operated to allow the extractor catheter 332 to adjust as needed along the changing diameter of the vasculature. In other instances, the handle 302 may be operated to restrict or prevent the extractor catheter 332 from changing size or shape. In other instances, the handle 302 may be operated to control the adjustment of the extractor catheter 332.

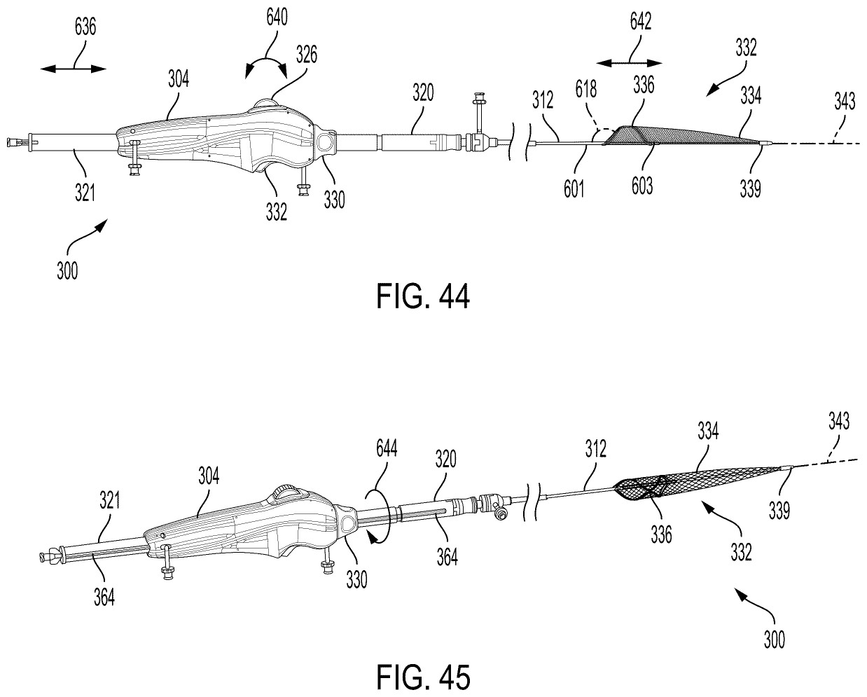

FIG. 44 is a side view of the extractor device 300. With the trigger 322 at rest, the distal tip 339 of the extractor catheter 332, which is attached to the inner shaft 308, is fixed in place along the axis 343 extending through a center of each of the inner shaft 308, the middle shaft 310, and the outer shaft 312. When the trigger 322 is depressed, the sliding member 320, and therefore the distal tip 339, are free to translate along the axis 343 in one of the directions of the double-sided arrow 642. For example, holding the trigger 322 depressed allows the extractor catheter 332 to adjust shape as the extractor catheter 332 is withdrawn through a blood vessel of a patient based on changes in size of the blood vessel. In another example, a physician may depress the trigger 322 and manipulate the shape of the extractor catheter 332 by manually translating the control portion 321 of the sliding member 320 along the axis 343 in one of the directions of the double-sided arrow 636. For instance, retracting the inner shaft 308 into the middle shaft 310 shortens a length of the mesh 334, which can allow for easier cleaning of clot inside the mesh 334. Shortening the mesh 334 also allows advancing the extractor catheter 332 closer to an inferior vena cava (IVC) filter prior to capturing clot material with the extractor catheter.

With the dial 326 at rest, the distal end of the coring element is free, and more specifically the distal ends 630C, 630D of the distal portion of the coring element 336 attached to the middle shaft 310 are free, to translate along the axis 343 in one of the directions of the double-sided arrow 642. For example, leaving the dial 326 disengaged allows the shape of the extractor catheter 332 to adjust as the extractor catheter 332 is withdrawn through, e.g., a blood vessel of a patient based on a change or changes in size of the blood vessel. When the dial 326 is depressed, the distal ends 630C, 630D are fixed in place along the axis 343. When the depressed dial 326 is rotated in one of the directions of the double-sided arrow 640, the position of the middle shaft 310, by virtue of the respective positions of the distal ends 630C, 630D, is finely controlled by the motion of the dial 326. For example, a physician may want fine control over the size of the mouth 335 of the mesh 334.

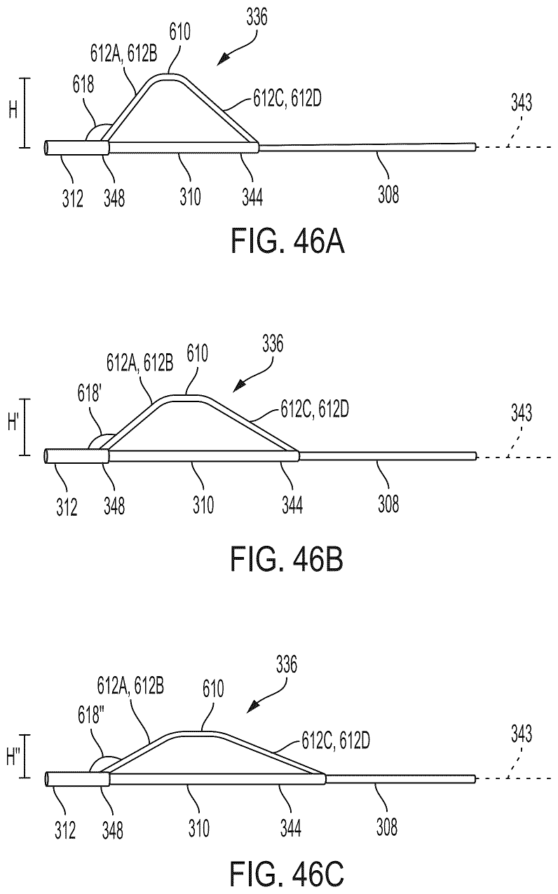

FIGS. 46A to 46C are a series of simplified depictions showing various shapes of the coring element 336. The distal ends of the third and fourth sets of arms 612C, 612D of the coring element 336 are coupled to the distal end 344 of the middle shaft 310 and the distal ends of the first and second sets of arms 612A, 612B of the coring element 336 are coupled to the distal end 348 of the outer shaft 312. FIG. 46A depicts a first shape of the coring element 336 in which the first and second sets of arms 612A, 612B form the angle 618 with the third plane including the x- and y-axes. The coring element 336 also has a height H in the first shape. The height H is measured between the axis 343 and the bridge 610 of the coring element 336.

A position of the distal end 348 of the outer shaft 312, and thereby the distal end of the coring element 336, is fixed translationally relative to the inner shaft 308 and the middle shaft 310. In this way, movement of the coring element 336 or of the middle shaft 310 can transition the coring element 336 into a second shape depicted in FIG. 46B. For example, when force applied to the bridge 610 toward the middle shaft 310 (e.g., by a vessel wall) increases as the diameter of the vessel decreases, the increase in force translates the distal end 344 of the middle shaft 310 along the axis 343 distally, i.e., away from the distal end 348 of the outer shaft 312. In the second shape of the coring element 336, the angle 618′ is greater than the angle 618 and the height H′ is less than the height H.

Movement of the coring element 336 or of the middle shaft 310 can further transition the coring element 336 into a third shape depicted in FIG. 46C. For example, as the vessel diameter decreases further, the force applied to the bridge 610 may increase further, which translates the distal end 344 of the middle shaft 310 farther along the axis 343 away from the distal end 348 of the outer shaft 312. In the third shape of the coring element 336, the angle 618″ is greater than the angle 618′ and the height H″ is less than the height H′. Each of the angles 618, 618′, 618″ may be within the ranges provided above for the angle 618.

It will be appreciated that when the mouth 335 of the mesh 334 is coupled to an arm of each of the first and second sets of arms 612 A, 612 B, the mouth 335 may be associated with each of the angles 618, 618′, 618″ and each of the heights H, H′, H″ as well, meaning the values of those angles and/or those heights may be the same, or at least substantially the same, for the mouth (and therefore the open end) of the mesh as for the coring element. An advantage of the coring element 336 is that flexion and relaxation of the coring element 336 has little to no effect on the size of the mouth 335 of the extractor catheter 332. Rather, the mouth 335 remains approximately the same size (e.g., the area circumscribed by the mouth remains approximately the same) despite the change in angles 618, 618′, 618″ and heights H, H′, H″. In this way, a mouth 335 that is similarly sized for accepting clot into the mesh 334 can be deployed throughout any of the vasculature diameters from the IVC bifurcation to the common iliac vessel to the popliteal vessel.

Another operation of controlling the extractor catheter 332 with the handle 302 involves rotating the extractor catheter 332. Specifically, rotating the knob 330 about the axis 343 in the direction of the arrow 644, as shown in FIG. 45, rotates the extractor catheter 332 about the axis 343 in the direction of the arrow 644. For instance, rotating the knob 330 rotates the sliding member 320, which rotates the inner shaft 308 and also the middle shaft hub 324 and the outer shaft hub 328, which respectively rotates the middle shaft 310 and the outer shaft 312. Rotation of the shafts 308, 310, 312 rotates the extractor catheter 332. In various embodiments, the knob 330 may be rotated in four 90° increments, though other suitable quantities of increments may be used in other embodiments.

A physician using the extractor device 300 may desire to change the orientation of the extractor catheter 332 so as to target different portions of the circumference of a blood vessel when breaking down and separating wall-adherent blood clots. In at least some embodiments, the extractor catheter 332 is only rotated when the extractor catheter 332 is outside of a body of a patient. For example, a physician may perform a coring action by withdrawing the extractor catheter 332 through a blood vessel and out of the vasculature, clean the extractor catheter 332 of the captured blood clots, rotate the orientation of the extractor catheter, and reintroduce the extractor catheter into the vasculature to perform another coring action. By rotating the extractor catheter 332 independent from rotation of the handle 302, the physician can maintain the handle 302 in an upright position, which enhances the ease of use of the extractor device 300.

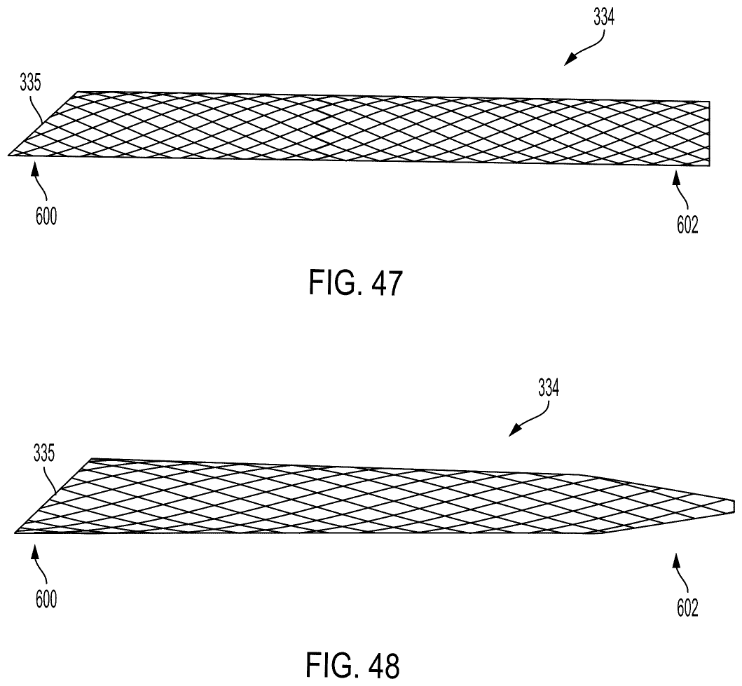

Example configurations of the mesh 334 will now be described. Referring to FIG. 47, in some embodiments, the mesh 334 may have a straight profile along a length of the mesh 334 such that a diameter of the mesh 334 adjacent to the mouth 335 is substantially equal to a diameter of the distal end 602 of the mesh 334. In such embodiments, the distal end 602 of the mesh 334 is bunched up and positioned within the distal tip 339 during assembly of the extractor device 300.

Referring to FIG. 48, in some embodiments, the mesh 334 may have a tapered profile along its length such that a diameter of the mesh 334 adjacent to the mouth 335 is greater than a diameter of the distal end 602 of the mesh 334. In such embodiments, less material needs to be bunched up within the distal tip 339 compared to the mesh 334 having the straight profile. In an example, the mesh 334 having the tapered profile can be made, in part, by braiding the mesh 334 on a component machined into the shape of the tapered profile.

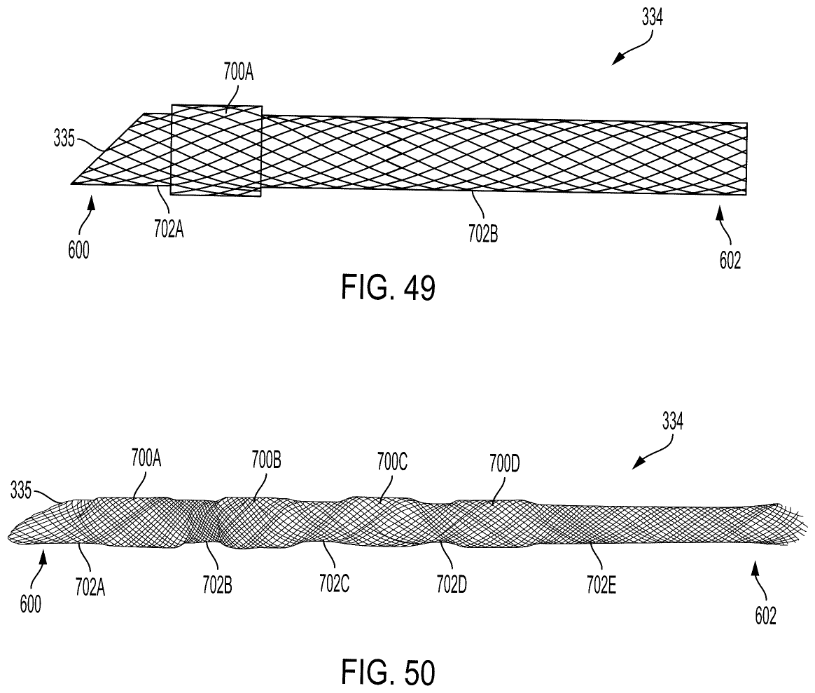

Referring to FIG. 49, in some embodiments, the mesh 334 may have a straight profile that includes a wider portion 700A (e.g., a bump). The bump 700A has a greater transverse dimension (e.g., diameter) than portions 702A, 702B (i.e., portions proximal and distal of bump 700A, respectively) of the mesh 334. In some aspects, the portions 702A and 702B have substantially equal diameters. The portion 702A includes the proximal end 600 of the mesh 334, and the portion 702B includes the distal end 602 of the mesh 334. In some embodiments, the portion 702B may have a tapered profile similar to the tapered profile of FIG. 48. The bump 700A can have a variety of widths or diameters in various aspects, and can also be disposed at various positions between the proximal end 600 and the distal end 602 of the mesh 334.

In some embodiments, the mesh 334 may include more than one bump 700A. For example, a mesh 334 with a length within a range of 14-18 cm may include four bumps, which may be of similar size and shape. FIG. 50 shows such a mesh 334 having a straight profile including four bumps 700A, 700B, 700C, and 700D. Portions 702A, 702B, 702C, 702D, and 702E of the mesh 334 have a smaller diameter than do the bumps 700A-700D. In some aspects, the portions 702A-702E each have substantially equal diameters. The portion 702A includes the proximal end 600 of the mesh 334, and the portion 702E includes the distal end 602 of the mesh 334. In some embodiments, the portion 702E may have a tapered profile similar to the tapered profile of the embodiment of mesh 334 of FIG. 48.

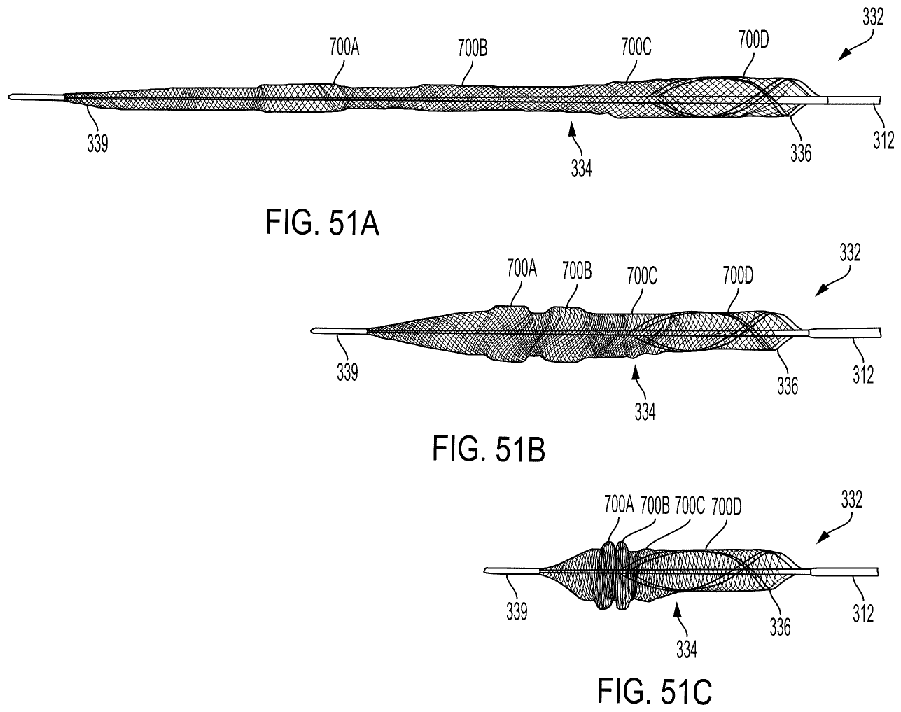

The one or more bumps 700A-700D enable the mesh 334 to compress axially without an outermost diameter (e.g., height) of the mesh 334 substantially increasing. For example, FIGS. 51A to 51C show three snapshots of the mesh 334 transitioning from an extended state to a compressed state. In FIG. 51A, the mesh 334 is shown in the extended state in which it is stretched and the outermost diameters of the bumps 700A, 700B, and 700C are less than the outermost diameter of the bump 700D. For example, to achieve the extended state, the inner shaft 308 was advanced relative to the middle shaft 310 and to the outer shaft 312. In FIG. 51B, the mesh 334 is shown in an intermediate state in which the outermost diameters of the bumps 700A and 700B are greater than the outermost diameters of the bumps 700C and 700D. For example, the inner shaft 308 was retracted relative to the middle shaft 310 and to the outer shaft 312 to transition the mesh 334 from the extended state to the intermediate state.

In FIG. 51C, the mesh 334 is shown in a compressed state in which the mesh is crunched into a compact shape and the outermost diameters of the bumps 700A, 700B, and 700C are marginally greater than the outermost diameter of the bump 700D. For example, the inner shaft 308 was retracted further relative to the middle shaft 310 and to the outer shaft 312 to transition the mesh 334 from the intermediate state to the compressed state. As shown in FIG. 51C, the outermost diameters of the bumps 700A and 700B did not substantially increase from the extended state to the compressed state while the length of the mesh 334 substantially decreased. The lack of substantial diameter increase is due, in part, to the material of one or more of the portions 702B-702D of the mesh 334 rolling within one or more of the bumps 700A-700D as the mesh 334 compresses.

Multiple advantages result from being able to compress the mesh 334 into a substantially shorter length without substantially increasing the outermost diameter of the mesh 334. For example, the compressed state of the mesh 334 enables advancing the extractor catheter 332 closer to an IVC filter prior to beginning the clot extraction process, allowing an operator to extract a greater amount of clot during a clot extraction procedure. In another example, the mesh 334 must be cleaned of clot between passes of the extractor device 300 and it can be easier to clean the mesh 334 when it is in the crunched, compact shape of the (or another similar) compressed state. For instance, the distal end 602 of the mesh 334 can be reached more easily. The diameters and widths referenced with respect to the structures shown in FIGS. 47 to 51 are examples of transverse dimensions taken perpendicular to the axis of the extractor catheter (e.g., axis 343, shown elsewhere) or to any of the shafts.

Performance of the mesh 334 compressing into the crunched, compact shape can vary based on the shape and dimensions of the bumps 700A-700D, the portions 702B-702D, and the transitions between adjacent ones of bumps 700A-700D and portions 702B-702D. For instance, if the bumps 700A-700D are too pronounced (e.g., too great of height, too steep of transition angle, etc.) relative to the portions 702B-702D, it may become difficult for the material of one or more of the portions 702B-702D of the mesh 334 to roll up within one or more of the bumps 700A-700D as the mesh 334 compresses. In an example, each of the bumps 700A-700D may have a length within a range of 1 to 2 centimeters (cm). In another example, each of the portions 702B-702D may have a length within a range of 1 to 2 centimeters (cm). In some embodiments, a length of at least one of the bumps 700A-700D may be equal to a length of at least one of the portions 702B-702D. In some embodiments, a length of each of the bumps 700A-700D may be equal to a length of each of the portions 702B-702D. In another example, there may be about a 2 millimeter (mm) (e.g., 2 mm) transition lengthwise between a bump 700A-700D and an adjacent portion 702A-702E that allows the diameter of the mesh 334 to gradually change.

The embodiment of mesh 334 having the one or more bumps 700A-700D can, in an example, be made, in part, by braiding the mesh 334 on a component machined into the shape of the profile that includes the one or more bumps 700A-700D. In at least some embodiments, making the mesh 334 can further include heat treating the mesh 334 to set the shape.

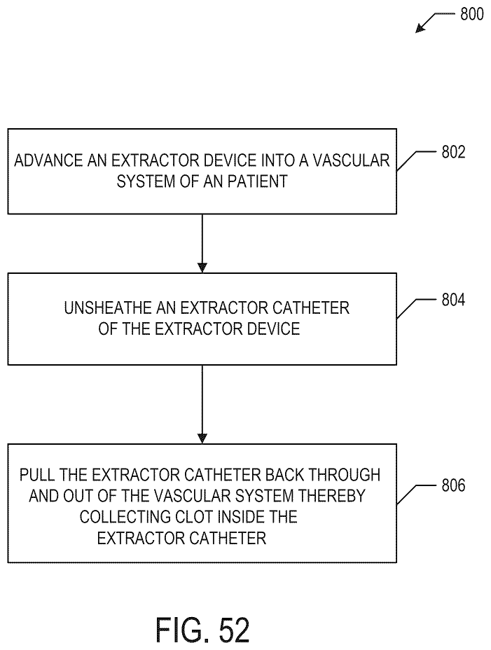

FIG. 52 is a flow chart of a method 800 for extracting clot from a vascular system of a patient. Method 800 includes, at block 802, advancing an extractor device (e.g., extractor device 300) into the vascular system of the patient. For instance, the distal tip 339 of the extractor device 300 may be inserted into a blood vessel and advanced further into the vascular system. For example, the distal tip 339 may be inserted into the mid popliteal vein at the back of a leg of the patient. The distal tip 339 may be inserted into the mid popliteal vein through an introducer, such as the introducer sheath 20.

In at least some embodiments, the distal tip 339 is advanced to the IVC bifurcation or iliac of the patient's vascular system, though another suitable position within the vascular system may be desired. A guidewire may be used to help advance the distal tip 339, which is configured with a lumen through which an appropriately-sized guidewire can extend. During insertion and advancement of the extractor device 300, the extractor catheter 332 is sheathed within the cover sleeve 314. It should be appreciated that the handle 302 and the cover sleeve hub 315 always remain outside of the patient's vascular system.

At block 804, with the distal tip 339 in the desired position, the extractor catheter 332 is unsheathed. For example, the cover sleeve 314 is manually pulled back to expose the extractor catheter 332. When exposed, the extractor catheter 332 expands into its pre-set shape. In some embodiments, the relevant features of the handle 302 are in positions that allow for such expansion automatically. In some embodiments, a length of the extractor catheter 332 (e.g., a length of the mesh 334) may be decreased manually. For example, a physician may depress the trigger 322 of the handle 302 and manually pull back the sliding member 320 to shorten the length of the mesh 334. With the shortened length of the mesh 334, the extractor catheter 332 may be advanced further into the vascular system, such as closer to an IVC filter. In some embodiments, the physician may manipulate the extractor catheter 332, such as to more closely align the shape of the extractor catheter 332 with the vessel. For example, the physician may depress and rotate the dial 326 to manipulate the extractor catheter 332.

At block 806, the extractor catheter 332 is then pulled back through and out of the vascular system to core (e.g., with the coring element 336) and collect clot (e.g., a length of clot) that can fit into the mesh 334. While the extractor catheter 332 is pulled back through the vascular system, the handle 302 may be operated to manipulate the extractor catheter 332 or to allow the extractor catheter 332 to adjust freely to changes in the vessels, such as the decrease in diameter from the iliac vessel to the popliteal vessel (e.g., from 16 mm down to 6 mm). For example, a physician may hold the trigger 322 depressed to allow the distal tip 339 to move freely to adjust a length of the mesh 334 as needed. In another example, the physician may refrain from depressing the dial 326 to allow the coring element 336 to freely adjust to the decreasing vessel diameter. In another example, the physician may depress and rotate the dial 326 to elongate (e.g., reduce height H) of the coring element 336 if the physician feels resistance from the vessel wall.

Once the extractor catheter 332 is removed from the vascular system of the patient, the mesh 334 may be cleaned of the captured clot and the method 800 can be repeated until the vessel(s) is cleared of clot. In some embodiments, the length of the mesh 334 may be shortened for ease of cleaning. In some embodiments, an orientation of the extractor catheter 332 relative to the handle 302 may be changed prior to reintroducing the extractor device 300 into the vascular system. For example, the physician may rotate the knob 330 of the extractor device 300 to rotate the extractor catheter 332 a desired amount.

It is noted that the order of one or more blocks (or operations) described with reference to FIGS. 14 and 52 may be changed, certain blocks may be combined with other blocks, additional blocks may be added, and some of the block may be omitted. It is also noted that one or more blocks (or operations) described with reference to FIG. 14 may be combined with one or more blocks (or operations) described with reference to FIG. 52.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A clot extraction device controller comprising:
a first shaft;
a second shaft disposed around a portion of the first shaft; and
a control handle including:
a housing;
a sliding member disposed through the housing and coupled to the first shaft;
a first hub disposed within the housing and coupled to the second shaft, wherein the sliding member is disposed through a portion of the first hub; and
a first control member coupled to the housing and configured such that the first control member is movable between a first position in which the first control member restricts movement of the sliding member relative to the housing and a second position in which the first control member allows movement of the sliding member relative to the housing.

2. The clot extraction device controller of claim 1, wherein the sliding member and the first hub are configured such that the sliding member may translate relative to the first hub.

3. The clot extraction device controller of claim 1, wherein the sliding member and the first hub are configured such that the sliding member and the first hub are restricted from rotating relative to one another.

4. The clot extraction device controller of claim 1, wherein the first control member is biased toward the first position of the first control member.

5. The clot extraction device controller of claim 1, wherein the control handle further includes a second control member and a second hub, the second control member configured and coupled to the housing such that the second control member is movable between a first position in which the second control member allows movement of the second hub relative to the housing and a second position in which the second control member restricts movement of the second hub relative to the housing.

6. The clot extraction device controller of claim 5, wherein the second control member is biased toward the first position of the second control member.

7. The clot extraction device controller of claim 1, further comprising a third shaft disposed around a portion of the second shaft, wherein:
the control handle further includes a second hub disposed within the housing and coupled to the third shaft; and
the sliding member is disposed through a portion of the second hub.

8. The clot extraction device controller of claim 1, further comprising a plurality of flushing ports.

9. A clot extraction device controller comprising:
a first shaft;
a second shaft disposed around a portion of the first shaft; and
a control handle including:
a housing;
a sliding member disposed through the housing and coupled to the first shaft;
a first hub disposed within the housing and coupled to the second shaft, wherein the sliding member is disposed through a portion of the first hub; and
a first control member coupled to the housing and configured such that the first control member is movable between a first position in which the first control member allows movement of the first hub relative to the housing and a second position in which the first control member restricts movement of the first hub relative to the housing.

10. The clot extraction device controller of claim 9, wherein the first control member is biased toward the first position of the first control member.

11. The clot extraction device controller of claim 9, wherein the first hub includes a first hub engagement portion and the first control member includes a first control member engagement portion configured to engage with the first hub engagement portion when the first control member is in the second position, thereby restricting movement of the first hub relative to the housing.

12. The clot extraction device controller of claim 11, wherein the first hub engagement portion includes one or more male and/or female teeth, the first control member engagement portion includes one or more male and/or female teeth, and, when the first control member engagement portion engages with the first hub engagement portion, one of the one or more male and/or female teeth of the first control member engagement portion engages one of the one or more male and/or female teeth of the first hub engagement portion.

13. The clot extraction device controller of claim 9, wherein the first hub includes a first plurality of teeth and the first control member includes a second plurality of teeth configured to engage with the first plurality of teeth when the first control member is in the second position, thereby restricting movement of the first hub relative to the housing.

14. The clot extraction device controller of claim 13, wherein:
the first control member includes a dial that includes the second plurality of teeth; and
the first control member is configured such that, when the first control member is in the second position, rotating the dial moves the first hub relative to the housing.

15. The clot extraction device controller of claim 9, wherein:
the first control member includes a dial coupled to an arm pivotable about a first axis relative to the housing; and
the dial is rotatable about a second axis relative to the arm.

16. The clot extraction device controller of claim 9, further comprising a second control member, wherein the second control member, the sliding member, and the first hub are configured such that the second control member, the sliding member, and the first hub are rotationally coupled to one another such that rotating the second control member rotates both the sliding member and the first hub.

17. The clot extraction device controller of claim 9, further comprising a plurality of flushing ports.

18. A clot extraction device control handle comprising:
a control member;
a sliding member configured to be coupled to an inner shaft;
a first hub configured to be coupled to a middle shaft disposed around a portion of the inner shaft; and
a second hub configured to be coupled to an outer shaft disposed around a portion of middle shaft;
wherein each of the control member, the first hub, and the second hub are rotationally coupled to the sliding member such that rotating the control member rotates each of the sliding member, the first hub, and the second hub.

19. The clot extraction device control handle of claim 18, wherein the sliding member is configured to translate axially relative to the first hub and the second hub.

20. The clot extraction device control handle of claim 18, wherein the sliding member is disposed through a portion of the first hub and a portion of the second hub.

21. The clot extraction device control handle of claim 18, wherein:
the sliding member includes a track;
the first hub includes a first coupling member disposed within the track; and
the second hub includes a second coupling member disposed within the track.

22. The clot extraction device control handle of claim 18, wherein:
the sliding member defines a channel;
the first hub includes a first elongated member disposed within the channel of the sliding member; and
the second hub includes a second elongated member disposed within the channel of the sliding member.

23. The clot extraction device control handle of claim 21, wherein the second coupling member is further disposed within a slot of the control member.

24. The clot extraction device control handle of claim 18, further comprising a plurality of flushing ports.
